SEQUENCE LISTING (5 sequences)

SEQ ID NO: 1 - DNA, 15 bp, Artificial Sequence
  Complementary sequence to a sequence that can assume a dissociative conformation
cccacccaccctccc

SEQ ID NO: 2 - DNA, 15 bp, Artificial Sequence
  Complementary sequence to a sequence that can assume a dissociative conformation
ccctcccacccaccc

SEQ ID NO: 3 - DNA, 16 bp, Artificial Sequence
  Sequence that can assume a dissociative conformation
gggtgggtgggtgggt

SEQ ID NO: 4 - DNA, 40 bp, Artificial Sequence
  Nucleic acid construct for amplification to determine presence of a target nucleic acid
gggagggcgggtgggttttttttttttttggcccgccctc

SEQ ID NO: 5 - DNA, 23 bp, Artificial Sequence
  Target nucleic acid
ccaaaaaaaaaaaaaacccaasd

(19) United States
(12) Patent Application Publication
(10) Pub. No.: US 2014/0051086 A1
(43) Pub. Date: Feb. 20, 2014

(54) Isothermal Amplification of Nucleic Acid

(71) Applicant: Besik Kankia, Columbus, OH (US)

(72) Inventor: Besik Kankia, Columbus, OH (US)

(73) Assignee: OHIO STATE INNOVATION FOUNDATION, Columbus, OH (US)

(21) Appl. No.: 14/006,416

(22) PCT Filed: Mar. 21, 2012
(86) PCT No.: PCT/US12/29880

§ 371 Date: Sep. 20, 2013

Related U.S. Application Data
(60) Provisional application No. 61/454,705, filed on Mar. 21, 2011.
(60) Provisional application No. 61/478,272, filed on Apr. 22, 2011.
(60) Provisional application No. 61/554,203, filed on Nov. 1, 2011.

(51) Int. Cl. C12Q 1/68

(52) U.S. Cl. CPC C12Q 1/6853
USPC 435/6.12; 536/23.1; 536/24.33

(57) ABSTRACT

An amplification system that provides methods and reaction components that allow for completely isothermal amplification for detection of target nucleic acid 24; allow non-enzymatic amplification for detection of target nucleic acid 24; and can be used to identify amplicons without having to create separate individual probes for each target nucleic acid 24.

### CROSS-REFERENCE TO RELATED APPLICATIONS

This application is a U.S. national phase filing under 35 U.S.C. §371 of International Patent Application No. PCT/US2012/029880, filed Mar. 21, 2012, entitled “Isothermal Amplification of Nucleic Acid,” which claims priority to and the benefit of the filing date of U.S. Patent Application Ser. No. 61/454,705, entitled “Isothermal Amplification of Nucleic Acid,” filed on Mar. 21, 2011; U.S. Patent Application Ser. No. 61/478,272, entitled “Isothermal Amplification of Nucleic Acid,” filed on Apr. 22, 2011; and U.S. Patent Application Ser. No. 61/554,203, entitled “Isothermal Amplification of Nucleic Acid,” filed on Nov. 1, 2011, the disclosures of which are incorporated by reference herein in their entireties.

### FIELD OF THE INVENTION

The present invention relates generally to detection and/or identification of nucleic acids via an amplification process, and more specifically to novel methods and components for detection and/or identification of nucleic acids via an amplification process.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Identification of a particular nucleic acid (e.g., via detection of the presence of a particular nucleic acid sequence—the “target nucleic acid”) is desirable for many reasons, including use in diagnostic applications, forensic applications, etc. However, often the target nucleic acid sequence may be only a small portion of the DNA or RNA in question, and/or the quantity of DNA or RNA may be limited so that it may be difficult to detect the presence of the target sequence using probes (such as oligonucleotide probes). Much effort has been expended in increasing the sensitivity of the probe detection systems, and processes have also been developed for amplifying the target sequence so that it is present in quantities sufficient to be readily detectable.

One such amplification method is the polymerase chain reaction (PCR). Since its initial design by Kary Mullis in 1984, PCR has impacted nearly every field in molecular biology, genetics, and forensic science. PCR is a technique to amplify a single or few copies of a particular nucleic acid sequence, e.g., DNA, across several orders of magnitude, thereby generating thousands to millions of copies of the sequence. PCR is the main tool presently used to amplify nucleic acid and study gene expression.

PCR relies on “thermal cycling,” which includes cycles of repeated heating and cooling of the DNA and other reaction components to cause DNA denaturation (i.e., separation of the double-stranded DNA into its sense and antisense strands) followed by enzymatic replication of the DNA. The other reaction components include short oligonucleotide DNA fragments known as “primers,” which contain sequences complementary to at least a portion of the DNA sequence associated with the target nucleic acid, and a DNA polymerase. These are components that facilitate selective and repeated amplification of the target sequence. As PCR progresses, the DNA generated is itself used as a template for further replication in subsequent cycles, creating a chain reaction in which the target DNA sequence is exponentially amplified.

More specifically, the DNA polymerase used in PCR is thermostable (and thus avoids enzyme denaturation at high temperatures) and amplifies target DNA by in vitro enzymatic replication. One such thermostable DNA polymerase is Taq polymerase, an enzyme originally isolated from the bacterium Thermus aquaticus. The DNA polymerase enzymatically assembles a new DNA strand from deoxynucleoside triphosphates (dNTPs) by using the denatured single-stranded DNA as a template. As is known to those of ordinary skill in the art, a deoxynucleoside triphosphate is deoxyribose having three phosphate groups attached, and having one base (adenine, guanine, cytosine, thymine) attached. However, as used herein, it will be recognized by those of ordinary skill in the art that arsenic may be substituted for phosphorous in the triphosphate backbone of the dNTP. The initiation of DNA synthesis and the selectivity of PCR results from the use of primers that are complementary to the DNA region targeted for amplification under specific thermal cycling conditions.

Thus, a basic PCR set up includes multiple components. These include: (1) a DNA template that contains the target DNA region to be amplified; (2) primers that are complementary to the 3′ ends of each of the sense strand and antisense strand of the target DNA; (3) a thermostable DNA polymerase such as Taq polymerase; and (4) dNTPs, the building blocks from which the DNA polymerases synthesizes a new DNA strand. Additionally, the reaction will generally include other components such as a buffer solution providing a suitable chemical environment for optimum activity and stability of the DNA polymerase, divalent cations (generally magnesium ions), and monovalent cation potassium ions (K⁺).

PCR is commonly carried out in a reaction volume of 10-200 μl in small reaction tubes (0.2-0.5 ml volumes) in an apparatus referred to as a thermal cycler. The thermal cycler heats and cools the reaction tubes to achieve the temperatures required at each of the following steps of the reaction:

Denaturation Step:

This step consists of heating the reaction to usually around 94-98° C. for approximately 20-30 seconds. It causes denaturation of the DNA template by disrupting the hydrogen bonds between complementary bases, yielding single strands of DNA.

Annealing Step:

The reaction temperature is lowered to usually around 50-65° C. for approximately 20-40 seconds allowing annealing of the primers to the single-stranded DNA template. Stable DNA-DNA hydrogen bonds are formed when the primer sequence closely matches the template sequence. The polymerase (e.g., Taq polymerase) binds to the primer-template hybrid and begins DNA synthesis.

Extension Step:

The temperature at this step depends on the DNA polymerase used. Taq polymerase has its optimum activity temperature at about 75° C., and commonly a temperature of 72° C. is used with this enzyme. At this step the DNA polymerase synthesizes a new DNA strand complementary to the DNA template strand by adding dNTPs that are complementary to the template in the 5′ to 3′ direction, condensing the 5′-phosphate group of the dNTPs with the 3′-hydroxyl group at the end of the extending DNA strand (as described above arsenic may substitute for phosphorous in a dNTP). The extension time depends on the DNA polymerase used and on the length of the DNA fragment to be amplified. Under optimum conditions, at each extension step the amount of the target DNA is doubled, leading to exponential amplification of the specific target DNA.

PCR usually includes of a series of 20 to 40 repeated cycles of the above-described denaturation, annealing, and extension steps. The cycling is often preceded by a single initialization step at a high temperature (>90° C.), and followed by one final hold at the end for final product extension or brief storage. The initialization step consists of heating the reaction to a temperature of usually 94-96° C. (or 98° C. if extremely thermostable polymerases are used), which is held for 1-9 minutes. The final hold usually occurs at 4-15° C. for an indefinite time and may be employed for short-term storage of the reaction. The temperatures used and the length of time they are applied in each cycle depend on a variety of parameters. These include the enzyme used for DNA synthesis, the concentration of divalent ions and dNTPs in the reaction, and the melting temperature (Tₘ) of the primers.

Following thermal cycling, agarose gel electrophoresis may be employed for size separation of the PCR products to check whether PCR amplified the target DNA fragment. The size(s) of the PCR products is determined by comparison with a molecular weight marker, which contains DNA fragments of known size, run on the gel alongside the PCR products. Probes may also be used to identify the presence of an amplified target DNA fragment (e.g., an oligonucleotide probe having a detectable label and a sequence complementary to the target nucleic acid sequence may be used; the probe will hybridize to target nucleic acid that is present and the label can be detected, thereby signifying the presence of the target nucleic acid).

There are many applications of PCR and its variants. For example, real-time PCR (RT-PCR) is an established tool for DNA quantification that measures the accumulation of DNA product after each round of PCR amplification. Thus, RT-PCR enables both detection and quantification of one or more specific sequences in a DNA sample (as absolute number of copies or relative amount when normalized to DNA input or additional normalizing genes). Such quantitative PCR methods allow the estimation of the amount of a given sequence present in a sample—a technique often applied to quantitatively determine levels of gene expression.

RT-PCR procedure follows the general principle of PCR. However, in RT-PCR, the amplified DNA is detected as the reaction progresses in real time (whereas in standard PCR, the product of the reaction is detected at the end of the reaction). One common method for detection of products in RT-PCR is the use of nonspecific fluorescent dyes that intercalate with double-stranded DNA (dsDNA). For example, SYBR Green is an asymmetrical cyanine dye that binds to dsDNA, and the resulting DNA-dye complex absorbs blue light (λₘₐₓ=488 nm) and emits green light (λₘₐₓ=522 nm). The DNA-binding dye, such as SYBR Green, binds to all dsDNA in PCR, causing fluorescence of the dye. An increase in DNA product during PCR therefore leads to an increase in fluorescence intensity and is measured at each cycle, thus allowing DNA concentrations to be quantified.

Another method for detection of products in RT-PCR is the use of sequence-specific DNA probes, which are oligonucleotides that are labeled with a fluorescent reporter that permits detection after hybridization of the probe with its complementary DNA target. Many of these probes include a DNA-based probe having a fluorescent reporter (e.g., at one end of the probe) and a quencher of fluorescence (e.g., at the opposite end of the probe). The close proximity of the reporter to the quencher prevents detection of its fluorescence; breakdown of the probe by the 5′ to 3′ exonuclease activity of the Taq polymerase breaks the reporter-quencher proximity and thus allows unquenched emission of fluorescence, which can be detected. An increase in the product targeted by the reporter probe at each PCR cycle therefore causes a proportional increase in fluorescence due to the breakdown of the probe and release of the reporter. Examples of such probes well known to those of ordinary skill in the art are molecular beacon probes, TaqMan® probes, and Scorpion™ probes.

Molecular beacons are single-stranded oligonucleotide probes that form a hairpin-shaped stem-loop structure. The loop contains a probe sequence that is complementary to a target sequence in the PCR product. The stem is formed by the annealing of complementary sequences that are located on either side of the probe sequence. A fluorophore and quencher are covalently linked to the ends of the hairpin. Upon hybridization to a target sequence the fluorophore is separated from the quencher and fluorescence increases. Hybridization usually occurs after unfolding of the hairpin and product duplexes in the denaturation step of the next PCR cycle.

TaqMan® probes are single-stranded unstructured oligonucleotides. They have a fluorophore attached to the 5′ end and a quencher attached to the 3′ end. When the probes are free in solution, or hybridized to a target, the proximity of the fluorophore and quencher molecules quenches the fluorescence. During PCR, when the polymerase replicates a template on which a TaqMan® probe is bound, the 5′-nuclease activity of the polymerase cleaves the probe. Upon cleavage, the fluorophore is released and fluorescence increases.

Scorpion™ probes use a single oligonucleotide that consists of a hybridization probe (stem-loop structure similar to molecular beacons) and a primer linked together via a non-amplifiable monomer. The hairpin loop contains a specific sequence that is complementary to the extension product of the primer. After extension of the primer during the extension step of a PCR cycle, the specific probe sequence is able to hybridize to its complement within the extended portion when the complementary strands are separated during the denaturation step of the subsequent PCR cycle, and fluorescence will thus be increased (in the same manner as molecular beacons).

With the above general background in mind, there are drawbacks to current PCR processes and probe technologies. For example, one of the factors limiting the yield of specific target nucleic acid is competition between primer binding and self-annealing of the product. At the initial stage of PCR, target molecules are at low enough concentrations that target self-annealing does not compete with primer binding and amplification thus proceeds at an exponential rate. However, with accumulation of target nucleic acid (e.g., DNA), self-annealing becomes dominant and PCR slows down and eventually amplification ceases.

Temperature cycling is another limitation of PCR since it requires expensive instrumentation for thermocycling, thereby complicating rapid detection of pathogens in the field and at point-of-care. In addition, rapid temperature changes facilitate mispriming of the target nucleic acid and affect stability of the polymerases.

To eliminate problems caused by temperature cycling, expensive instrumentation, competition between primer binding and undesired self-annealing of target DNA, and difficulty with point-of-care testing, further amplification methods and components (e.g., primers) have been recently developed (as described in U.S. Provisional Application Ser. No. 61/338,475 and International Application No. PCT/US2011/25411, the disclosures of both of which are incorporated herein in their entireties). These methods and components, in general, inhibit self-annealing by providing at least one primer (e.g., an oligonucleotide) for amplification of a target nucleic acid (e.g., DNA), wherein the primer is adapted to conform into a structure (e.g., a non-B DNA conformation or other DNA structure) in which intramolecular base-pairing allows or causes the primer to dissociate from a double-stranded DNA (which may be referred to herein as a “dissociative structure” or “dissociative sequence”). Such a structure may include triplexes or quadruplexes. The particular structure—e.g., a quadruplex—may form during an extension step of an amplification method, such as PCR. As this occurs, the primer necessarily separates from its binding site on the target DNA sequence while the extending portion (DNA polymerase adding dNTPs to the sequence) remains bound (at least temporarily) in a double-stranded configuration. This process may be referred to herein as “Dissociative Sequence Priming Amplification” or “Dissociative Structure Priming Amplification” (which may be referred to herein as “DSPA”). Other amplification processes may be compatible with DSPA. For example, helicase-dependent amplification (wherein a helicase is used to unwind the DNA duplex without having to alter the temperature of the reaction) may be compatible with DSPA (e.g., helicases may be used in the DSPA process).

By use of primers that form dissociative structures, an amplification process that is primarily isothermal is achieved. In other words, the process of amplification, once begun with DSPA primers, can be isothermal. This is because, as described above, when the primer forms its dissociative structure, it necessarily separates from its binding site on the target DNA sequence, and so this is achieved without having to raise the temperature of the reaction to denature the strands. However, as will be appreciated by those of ordinary skill in the art, naturally-occurring target DNA will not necessarily include a sequence complementary to the DSPA primer sequence (where the DSPA primer can bind to begin replication). And so, the DNA templates in these reactions must first have primer binding site (PBS) sequences added to the DNA template. This can be accomplished by 2 cycles of traditional PCR. Once accomplished, the DSPA primer(s) [such as quadruplex forming primer(s)] will be added to the mix and amplification may be continued under isothermal conditions. However, the use of these 2 cycles of traditional PCR prevents the method from being completely isothermal, and prevents the complete elimination of thermal cycling, equipment, etc., which is a barrier to point-of-care use.

Several other versions of isothermal DNA amplification have previously been developed [see Tomita, N. et al. (2008) Loop-mediated isothermal amplification (LAMP) of gene sequences and simple visual detection of products. Nature protocols, 3, 877-882; Vincent, M., et al. (2004) Helicase-dependent isothermal DNA amplification. EMBO reports, 5, 795-800; Andras, S. C. et al. (2001) Strategies for signal amplification in nucleic acid detection. Molecular biotechnology, 19, 29-44; Walker, G. T. et al. (1992) Strand displacement amplification—an isothermal, in vitro DNA amplification technique. Nucleic acids research, 20, 1691-1696; Walker, G. T. et al. (1992) Isothermal in vitro amplification of DNA by a restriction enzyme/DNA polymerase system. Proceedings of the National Academy of Sciences of the United States of America, 89, 392-396; Fox, J. D. et al. (2009) In Logan, J. et al. (eds.), Real-time PCR. Caister Academic Press, Norfolk, UK, pp. 163-175]. However, all these existing systems require extra reaction components or polymerases with specific activities, presenting drawbacks to these systems, as well.

In addition to the problems with developing a completely isothermal amplification process, there are other drawbacks to current PCR processes. For example, traditional PCR depends on the enzymatic activity of DNA polymerases. Such polymerization-based amplification can yield a macroscopically observable polymer, visible to the unaided eye, which is a preferable quality for point-of-care analysis. However, it requires coupling biotinylated dNTPs to DNA hybrids and requires 0.5 nM or higher concentrations of target nucleic acid, which reduces the usefulness of the test. Surface plasmon resonance coupled with interferometry can detect as little as 10 pM of target (thereby resolving the initial problem), but this requires instrumentation unsuitable for point-of-care diagnostics (thereby creating another problem). Gold nanoparticles can be visualized with the unaided eye at high pM to nM target concentrations. However, to increase sensitivity further, they must be coupled with PCR or other specialized detection platforms. A sandwich-type binding assay is able to detect 60 fmol target DNA, however it depends on biotinylated capture oligonucleotides, repeated washing steps and additional liposome components. Hybridization chain reaction and entropy-driven signal amplification do not require the use of specific detection platforms, and are based on autocatalytic reactions between DNA oligonucleotides in solution. However, both of those methods use complicated reactions and detection mechanisms. In addition, these methods suffer from low sensitivity due to significant levels of spontaneous autocatalysis even in the absence of target molecules. As a result, all of the above-described methods and variations are not suitable for point-of-care analysis.

Still further, there are drawbacks to the probes used to detect products in RT-PCR, such as sequence-specific DNA probes (e.g., molecular beacons, Taq Man®, and Scorpion™ probes). For example, there are several disadvantages with molecular beacons. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences, which introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, molecular beacons can't be used under isothermal conditions. Fifth, design of the probe requires considerable effort and knowledge of nucleic acid thermodynamics. And sixth, probe hybridization involves a bimolecular probe-primer system. This makes the reaction entropically unfavorable, slows down hybridization, and complicates product detection at exponential growth. The hybridization is much faster and efficient with a monomolecular probe-primer system [as described in Whitcombe, D. et al. (1999) Detection of PCR products using self-probing amplicons and fluorescence. Nat Biotechnol, 17, 804-807, incorporated by reference herein in its entirety].

All of the shortcomings listed above for molecular beacons hold true for TaqMan® probes. And, an additional disadvantage of TaqMan® probes is that they require the 5′-nuclease activity of the DNA polymerase used for PCR.

Additionally, many of the shortcomings listed for molecular beacons hold true for Scorpion™ probes. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences, which introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, Scorpion™ probes can't be used under isothermal conditions. And fifth, design of the probes requires considerable effort and knowledge of nucleic acid thermodynamics.

Further, fluorescent reporter probes do not prevent the inhibitory effect of primer dimers (i.e., sets primers that are complementary to one another, and thus hybridize to one another—forming a primer dimer—rather than hybridizing to the target template denatured DNA strands), which may depress accumulation of the desired products in the reaction.

Still another disadvantage of current detection mechanisms is that two separate functions, recognition and detection, are combined within a probe. For example, the traditional RT-PCR process includes: (i) recognition of target nucleic acid by primer(s); (ii) subsequent amplification; and then (iii) recognition of amplicons by a probe, which is accompanied by fluorescence reporting. Thus, in traditional RT-PCR recognition happens twice (primer recognition and probe recognition). The bifunctional nature of the probes (i.e., the probes provide both recognition and reporting) requires that the fluorophore-quencher pair be attached to each DNA probe sequence, which makes quantification impractical when several targets are tested. For example, to perform 96-well quantification using molecular beacons, it is necessary that the same fluorophore-quencher pair be attached to 96 different probes. This greatly increases the time, difficulty, and expense of such a test.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In an overarching aspect, the present invention provides an amplification system that reduces or eliminates the shortcomings of PCR described above. For example, various aspects of the present invention provide methods and reaction components that (i) allow for completely isothermal amplification for detection of target nucleic acid; (ii) allow non-enzymatic amplification for detection of target nucleic acid; and (iii) can be used to identify amplicons without having to create separate individual probes for each target nucleic acid.

Many aspects of the invention described herein relate to amplification using primers having dissociative sequences—DSPA. And so a brief description of that amplification process follows. However, while the DSPA process described below may, at times, refer to “quadruplexes” or other particular dissociative structures, it will be recognized by those of ordinary skill in the art that the invention is not limited to any particular sequence, or to a sequence that forms a quadruplex or any other particular structure, and that any sequence that dissociates from a DNA duplex to form another structure (whether quadruplex or a structure other than a quadruplex) may be used in accordance with the principles of the present invention. Further, as described above, other amplification processes may be compatible with DSPA. For example, helicase-dependent amplification (wherein a helicase is used to unwind the DNA duplex without having to alter the temperature of the reaction) may be compatible with DSPA (e.g., helicases may be used in the DSPA process).

As described above, PCR is limited by competition between primer binding and undesired self-annealing of target DNA. One aspect of DSPA inhibits self-annealing by providing at least one primer for amplification of a target nucleic acid (e.g., DNA), wherein the primer is adapted to conform into a dissociative structure—such as a quadruplex. The primer may conform into the dissociative structure during the extension step of PCR. As this occurs, the primer necessarily separates from its binding site on the target DNA sequence while the extending portion (DNA polymerase adding dNTPs to the sequence) remains bound (at least temporarily) in a double-stranded configuration.

Further, the primers used in DSPA may be universal. In other words, each primer in the primers used in the reaction includes the same sequence (or a substantially similar sequence that allows amplification to occur). As is known to those of ordinary skill in the art, in standard PCR at least two different primers (i.e., having two different sequences) are used (i.e., a first set of primers, wherein each primer of the first set includes the same or similar sequence, and a second set of primers wherein each primer of the second set includes the same or similar sequence, that sequence being different from the sequence of the primers in the first set). The need for the two sets of primers is to provide for amplification using each of the single strands from the DNA. Thus, one strand will be replicated using primers from the first set. The second strand (which is complementary to the first strand) will also be replicated. However, as that second strand is complementary to the first strand, the primers of the second set may be complementary to the primers of the first set. This creates the problem of primer-dimers (when the primers hybridize to one another—rather than to the target template denatured DNA strands). However, in DSPA, each of the primers used have the same or similar sequence. There is no second set of complementary primers that is needed. As such, there are no other complementary primers for the primers of the present application to hybridize to, thus eliminating the problem of primer dimers. Further, as the end being extended is currently bound with the target region, self-annealing of product is eliminated. And, the original primer binding site on the target DNA region is open for binding of another primer.

While “at least one primer” and a site being “open for binding of another primer” are discussed herein, it will be recognized by those of ordinary skill in the art that the “at least one primer” and the “another primer” may have the same sequence (or substantially similar sequence) as it is known that PCR employs multiple copies of a primer for amplification of a target DNA sequence. Further, as is known to those of ordinary skill in the art, the reaction components generally include multiple copies of primers. Thus, it will be understood that “at least one primer” or “one primer” or “a primer” or “the primer” or like references may refer to a single primer, or a primer among a set of multiple copies of the same or similar primers. Further, while various PCR procedures described herein are discussed as amplifying DNA, those of ordinary skill in the art will recognize that does not limit the disclosure to those seeking DNA sequences, as procedures such as reverse transcription PCR are well known, wherein reverse transcriptase reverse transcribes RNA into cDNA, which is then amplified by PCR.

Further, various nucleic acids, segments thereof, sequences, etc. are referred to herein as being “complementary” to one another. As used herein, “complementary” does not require an exact base-pair matching between each base of complementary sequences, for example. It only requires enough of a match that the sequences are capable of hybridizing.

Thus, the primer(s) in DSPA may be based on any sequence that is capable of forming a structure that allows or causes the primer to dissociate from a double-stranded DNA and form the particular structure—e.g., a quadruplex—such as during an extension step of PCR. One aspect of DSPA, then, uses the free energy of dissociative structures (such as quadruplexes) to drive unfavorable (endergonic) reactions of nucleic acids (e.g., isothermal PCR). One key point of these reactions is that some sequences—e.g., some G-rich sequences—are capable of forming structures/conformations with significantly more favorable thermodynamics than the corresponding DNA duplexes. The sequences are incorporated within DNA duplexes, which after interaction with an initiator (e.g., DNA polymerase) self-dissociate from the complementary strand and fold into their dissociative structures (e.g., quadruplexes). The energy of formation of these structures is used to drive PCR at substantially constant temperature.

Because DSPA inhibits product self-annealing and increases the number of PCR cycles within the exponential growth phase, the efficiency of PCR is improved by elongating the window of exponential amplification. And, since the dissociative structure is more stable than its corresponding duplex, unfolding of the duplex or release of target for the coming primers can occur without the need of substantial temperature change or any temperature change. In other words, in standard PCR, following the extension step, the DNA is in a duplex form. The next cycle then begins by raising the temperature to a point that the double-stranded DNA again denatures (i.e., separates into single strands). This is necessary in order to provide the separated sense and antisense DNA strands for primer binding (to each of the strands), followed by elongation during the next extension step (once the temperature of the reaction is reduced). However, by using primers based on the principles of DSPA, the primers and extending nucleotides that are added during the extension step naturally conform into the dissociative structure (such as a quadruplex). As this occurs, the primer (forming the dissociative structure) naturally separates from the target DNA sequence complementary to the primer, thereby leaving the target region complementary to the primer exposed in single-stranded form for binding of the next primer. This occurs without requiring raising of the temperature to denature the strands from one another. Thus, once begun, DSPA can proceed under isothermal conditions. As described above, isothermal DNA amplification is desirable because it would not require expensive instrumentation for thermocycling (as does standard PCR) and thus would allow for DNA amplification in the field and at point-of-care.

However, as described in the Background, DSPA is not completely isothermal due to the need to incorporate primer binding sites (PBS) into the target DNA, by using cycles of traditional PCR. One aspect of the present invention, then, provides an amplification process that is completely isothermal such that it would be suitable for point-of-care analysis.

In general, this aspect of the present invention includes at least one nucleic acid construct including first, second and third sequence segments. This construct may be used to identify target nucleic acid via an amplification process (in this process, it may be the construct itself that is amplified—with the amplification of the construct signifying the presence of target nucleic acid). At least a portion of the first sequence segment includes a sequence adapted to conform into a structure that dissociates from a complementary strand of a DNA duplex (i.e., a dissociative sequence adapted to form a dissociative structure). At least a portion of the second sequence segment includes a sequence that is complementary to a target nucleic acid. And, at least a portion of the third sequence segment includes a sequence that is complementary to the dissociative sequence portion of the first sequence segment. The nucleic acid construct may include a detectable label, so that the presence of the target nucleic acid can be confirmed, for example.

The nucleic acid construct may be in the form of a stem-loop. The stem region is formed by the dissociative-structure-forming sequence (i.e., first sequence segment) duplexed with its complementary strand (i.e., third sequence segment). The loop region includes the second sequence segment (which is complementary to a target nucleic acid sequence). Target nucleic acid binds to the loop region of the construct and unfolds it, which releases the third sequence segment from the first sequence segment. This frees the third sequence segment to be bound by primers (having a sequence complementary to the third sequence segment), thereby initiating the amplification reaction. In other words, the third sequence segment provides a PBS. Further, the dissociative-structure-forming sequence included in the first sequence segment of the nucleic acid is a sequence such as would be used for a primer in DSPA.

In order to perform the amplification reaction, then, the stem-loop nucleic acid construct described above is combined with target nucleic acid (or with a sample that one wants to test for the presence of a particular target nucleic acid) and at least one primer having a dissociative-structure-forming sequence as described in the first sequence segment (although these primers are free in solution and are not part of the stem-loop construct described above). Thus, when the target nucleic acid hybridizes with the loop region of the stem-loop construct, the stem-loop is unfolded. This unfolding releases the third sequence segment (having a PBS) from the first sequence segment. The first sequence segment will then form its dissociative structure (such as a quadruplex), and the PBS of the third sequence segment remains free for the primers in the mixture to bind thereto and start an amplification reaction. Once a primer attaches to the PBS, it replicates the unfolded loop portion (i.e., the probe) of the stem-loop construct during extension. As this occurs, the target nucleic acid will be released from the duplex of unfolded stem-loop and primer-extending-sequence, thereby allowing the target nucleic acid to be free for binding to another stem-loop construct. This approach has at least two advantages: (i) it allows design of a truly isothermal mechanism; and (ii) it can be used in detection of RNA pathogens, such as HIV, without reverse transcription.

Further, as described in the Background, PCR, which depends on the enzymatic activity of DNA polymerases, is not ideally suited for point-of-care use. Thus, another aspect of the present invention provides protein-free dissociative-structure-based amplification. In general, this aspect of the present invention provides a mixture of nucleic acid constructs including at least one first nucleic acid construct and at least one second nucleic acid construct. The at least one first nucleic acid construct and at least one second nucleic acid construct are designed such that they work in concert to provide an amplification reaction that can identify a target nucleic acid sequence (e.g., a DNA sequence) without the use of enzymes as in standard PCR. The reaction in this aspect of the present invention may also proceed isothermally.

To that end, the first nucleic acid construct includes a first sequence segment and a second sequence segment, wherein at least a portion of the first sequence segment includes a sequence adapted to conform into a structure that dissociates from a complementary strand of a DNA duplex, and wherein at least a portion of the second sequence segment includes a sequence that is complementary to a target nucleic acid. And, the second nucleic acid construct includes a first sequence segment and a second sequence segment, wherein at least a portion of the first sequence segment includes a sequence adapted to conform into a structure that dissociates from a complementary strand of a DNA duplex, and wherein at least a portion of the second sequence segment includes a sequence that is substantially similar to the target nucleic acid such that the second sequence segment of the second nucleic acid construct can bind with the second sequence segment of the first nucleic acid construct.

Like the nucleic acid construct for isothermal amplification and identification described above, each of the first and second nucleic acid constructs in this aspect of the present invention may be provided in the form of a stem-loop nucleic acid construct. In each of the stem-loop constructs, the portion of the sequence which includes a dissociative-structure-forming sequence provides a portion of the stem (being duplexed with a complementary sequence). A primary portion of the loop of the first nucleic acid construct includes a sequence that is complementary to the target nucleic acid, and a primary portion of the loop of the second nucleic acid construct includes a sequence that is substantially the same as the target nucleic acid sequence.

When the first and second nucleic acid constructs of this aspect of the present invention are combined with target nucleic acid, the target nucleic acid hybridizes with the loop segment of the first nucleic acid construct. This unfolds the stem-loop of the first nucleic acid construct, thereby unwinding the stem. Once unwound, the sequence of the now free dissociative-structure-forming sequence (i.e., the first sequence segment) forms its dissociative structure (e.g., a quadruplex). As a result, the DNA duplex between the loop portion and the target nucleic acid is destabilized and the complex quickly dissociates. The released target binds to another first nucleic acid stem-loop construct and repeats the same cycle.

Meanwhile, the denatured first nucleic acid construct, now having a dissociative structure at its 5′ end, binds to the stem-loop of the second nucleic acid construct [with hybridization between the second sequence segment of the first nucleic acid construct and the loop segment (second sequence segment) of the second nucleic acid construct]. This induces a similar unwinding/dissociation process in the second nucleic acid construct. Once unwound, the first sequence segment of the second nucleic acid construct forms its dissociative structure (e.g., a quadruplex). As a result, the DNA duplex between the first and second nucleic acid constructs is destabilized, and the two separate. The released second nucleic acid construct now binds to and unfolds stem-loop of another first nucleic acid by denatured second nucleic acid. At this point, the reaction becomes autocatalytic, i.e., the product of each cycle serves as the catalyst for the subsequent cycles. Thus, amplification occurs in the absence of any standard DNA polymerases, and can proceed isothermally.

Further, as described above, a drawback of current quantification systems is that they use FRET-based applications (Förster Resonance Energy Transfer), which require costly synthesis and considerable effort to design a sensitive probe. As is known to those of ordinary skill in the art, FRET is a mechanism describing energy transfer between two chromophores. A donor chromophore, initially in its electronic excited state, may transfer energy to an acceptor chromophore (in proximity, typically less than 10 nm) through nonradiative dipole-dipole coupling. Further, the processes currently used require multiple probes for multiple targets (i.e., one probe for each target), which greatly increases materials, time, and expense.

Thus, another aspect of the present invention provides FRET-based detection that increases the multiplex capability of DSPA. A fluorescent nucleotide donor is placed internally and a fluorescent acceptor is attached at 5′-end of a DSPA primer. The fluorescence emission peak of the donor overlaps the excitation peak of attached acceptor.

In another aspect of the invention, a nucleic acid construct may be provided that including multiple sequence segments, each having a detectable label, that allows for amplification of the signal generated.

More specifically, the nucleic acid construct may include (1) a first sequence strand, and (2) plurality of nucleotide segments, wherein the plurality of nucleotide segments each include a sequence that is complementary to at least a portion of the sequence of the first sequence strand. As a result, the plurality of nucleotide segments can act as a segmented version of a complementary strand, and, at least initially, retain the first sequence strand in a pseudo-duplex form (e.g., a duplex including one complete strand having bound thereto multiple fragments of a “second strand”).

The first sequence strand of nucleotides includes from the 5′ to the 3′ end: (1) a first segment having a sequence of nucleotides complementary to a target nucleic acid, and (2) a plurality of segments, each of the plurality of segments having a detectable label. Each of the plurality of segments is adapted to conform into a conformation having a free energy with more favorable thermodynamics than a corresponding B-DNA duplex. For example, each of the plurality of segments may be adapted to conform into a quadruplex.

As described above, the plurality of segments initially retains the first sequence strand in a pseudo-duplex form. This is accomplished because each of the plurality of nucleotide segments includes a sequence that is complementary to either (1) a sequence spanning the first segment and one of the plurality of segments of the first sequence strand, or (2) at least two of the plurality of segments of the first sequence strand.

When target nucleic acid hybridizes with its complementary part of the first sequence strand, the first of the plurality of segments is displaced. This is followed by first quadruplex (or other non-B-DNA duplex) formation, which in turn destabilizes next bimolecular duplex and so on. As the quadruplexes (or other non-B-DNA conformations) form, the labels are detectable, and the multiple labels provide and amplified signal.

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 is a graph showing a typical RT-PCR curve.

FIG. 2 is a schematic illustration of the DSPA process.

FIG. 3 shows the incorporation of the DSPA target site (dotted portion) in templates by attachment of quadruplex forming sequences (hash-marked portion) to primers.

FIG. 4A is a schematic of isothermal signal amplification showing an exponential growth pattern.

FIG. 4B is a schematic of isothermal signal amplification showing a linear growth pattern.

FIG. 5 is a schematic of a DNA G-quartet.

FIG. 6 is a schematic of structures and modes of action of previous probes with panel A showing a molecular beacon, panel B showing a TaqMan®, and panel C showing a Scorpion™ probe.

FIG. 7 is a schematic of non-enzymatic signal amplification.

FIG. 8 is a schematic showing an example of FRET between 2Ap and Alexa405 upon quadruplex formation.

FIG. 9 is a graph showing fluorescence melting curves of single-stranded oligonucleotides.

FIG. 10 shows fluorescence melting curves of G3T-ds15 duplex (i.e., GGG(2Ap)GGTGGGTGGG (“2Ap-G3T”) in duplex form with its complementary strand CCCACCCACCCTCCC [SEQ. ID. NO. 1]), wherein the black and squared lines correspond to heating and cooling (at 1° C./min rate), respectively.

FIG. 11 shows fluorescence melting curves of a 2Ap-G3T duplex in 15 mM KCl, 35 mM CsCl, 2 mM MgCl₂, 20 mM Tris-HCl, pH 8.7 wherein the black and squared lines correspond to heating and cooling (at 1° C./min rate), respectively.

FIG. 12 shows UV melting curves of G3T-ds15 and G3T-ds13 (i.e., GGG(2Ap)GGGTGGGTG in duplex form with a complementary strand CCCTCCCACCCACCC [SEQ. ID. NO. 2]) duplexes in the presence (-∘- and -□-) and absence (-Δ- and black line) of K⁺ ions.

FIG. 13 includes schematic diagrams showing two possible structures of (GGGT)₄ [SEQ. ID. NO. 3] with panel A showing an antiparallel conformation based on NMR work, and with panel B showing a parallel conformation suggested on the bases of thermodynamic and spectral studies.

FIG. 14 is fluorescence spectra of GGG(6MI)GGGCGGGCGGG without and with its complementary strand.

FIG. 15 is a schematic of a nucleic acid construct including multiple sequences having fluorescent nucleotides for multiplexing of signal.

FIG. 16 shows (1) in panel A, a sequence of an exemplary stem loop probe (the 5′ to 3′ sequence at the bottom of panel A, with primer binder site underlined with hash-marked line, loop portion underlined, and dissociative-structure-forming portion underlined by dotted segment) as would be used in linear amplification (as shown in FIG. 4B) unfolded and bound to target nucleic acid (underlined by dashed line in panel A) and primer (underlined by dotted segment of upper sequence in panel A); and (2) in panels B and C, graphs demonstrating that the stem loop probe of this embodiment leaks since its 3′ end is able to form a dissociative structure, such as a quadruplex.

FIG. 17 shows (1) in panel A, a sequence of an exemplary stem loop probe (the 5′ to 3′ sequence at the bottom of panel A, with primer binder site underlined with hash-marked line, loop portion underlined, and dissociative-structure-forming portion underlined by dotted segment) including one CC mismatch, which prevents the 3′ end from forming a dissociative structure (such as a quadruplex) and a primer GG mismatch at the 5′ end; and (2) in panels B and C, graphs which demonstrate that leakage (such as shown in FIG. 16) is reduced and eliminated by inhibiting the formation of the dissociative structure.

FIG. 18 is a schematic of isothermal signal amplification showing a linear growth pattern, and using an example of a stem loop probe such as that as shown in FIG. 17.

FIGS. 19A-C are schematics showing reaction mixtures, and demonstrating how DSPA simplifies the reaction mixture (with FIG. 19A showing the reaction mixture for typical PCR/immuno-PCR, FIG. 19B showing the reaction mixture for SLP (stem-loop probe)-DSPA, and FIG. 19C showing the reaction mixture for immuno-DSPA).

FIGS. 20A-D are schematics showing modularity of recognition and signal production using DSPA, and additionally showing the use of an applied magnetic field to progress nucleic acid adsorbed onto metal particles through solutions.

FIGS. 21A and B are schematics showing the universal primer/probe nature of DSPA in multi-well diagnostics, and the use of an applied magnetic field to progress nucleic acid adsorbed onto metal particles through solutions.

FIGS. 22A and B are schematics showing the monomolecular nature of detection using DSPA.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In an overarching aspect, the present invention provides an amplification system that reduces or eliminates the shortcomings of PCR described above. For example, various aspects of the present invention provide methods and reaction components that (i) allow for completely isothermal amplification for detection of target nucleic acid; (ii) allow non-enzymatic amplification for detection of target nucleic acid; and (iii) can be used to identify amplicons without having to create separate individual probes for each target nucleic acid.

Many aspects of the invention described herein relate to amplification using primers having dissociative sequences—DSPA—as well as other reaction components having dissociative sequences. And so a brief description of that amplification process follows. However, while the DSPA process described below may, at times, refer to “quadruplexes” or other particular dissociative structures, it will be recognized by those of ordinary skill in the art that the invention is not limited to any particular sequence, or to a sequence that forms a quadruplex or any other particular structure, and that any sequence that dissociates from a DNA duplex to form another structure (whether quadruplex or a structure other than a quadruplex) may be used in accordance with the principles of the present invention. Further, as described above, other amplification processes may be compatible with DSPA. For example, helicase-dependent amplification (wherein a helicase is used to unwind the DNA duplex without having to alter the temperature of the reaction) may be compatible with DSPA (e.g., helicases may be used in the DSPA process).

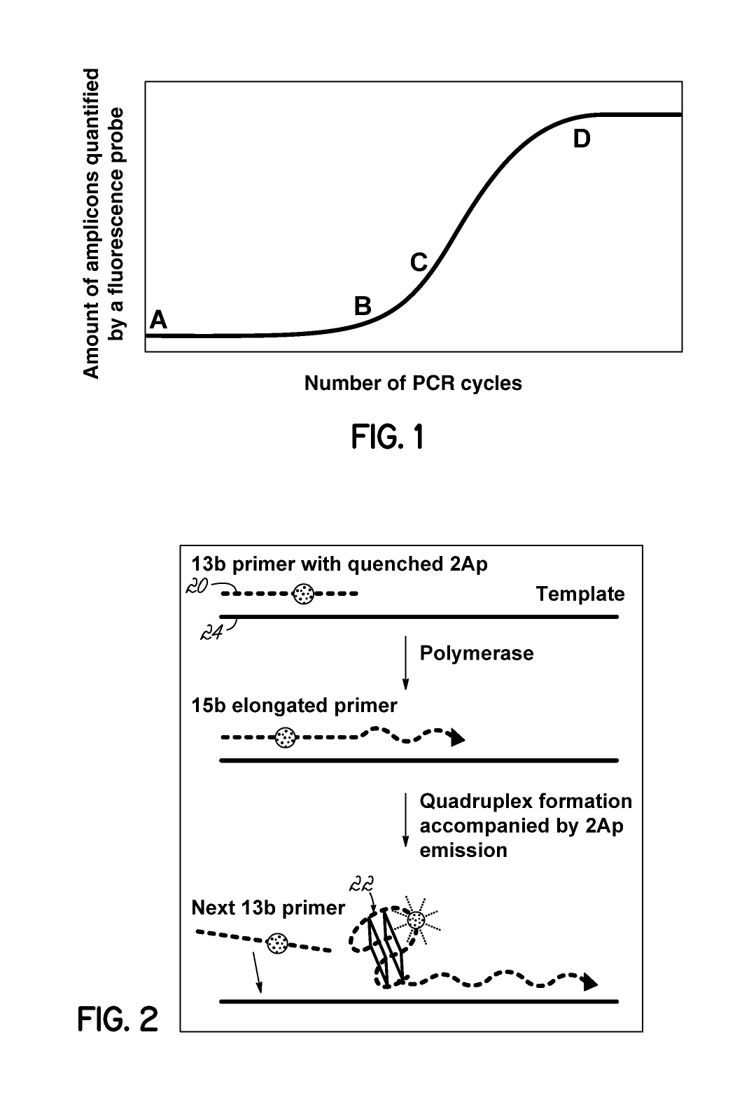

As described above, PCR is limited by competition between primer binding and undesired self-annealing of target DNA. Referring to FIG. 1, which shows a typical PCR, at the initial stage of PCR, product molecules are at low enough concentrations that product self-annealing does not compete with primer binding and amplification proceeds at an exponential rate (see the AC segment, FIG. 1; the AB segment corresponds to exponential phase undetectable by fluorescence measurements). However, with accumulation of product DNA, self-annealing becomes dominant and PCR slows (CD segment, FIG. 1) and eventually DNA amplification ceases (plateau, FIG. 1).

One aspect of DSPA inhibits self-annealing by providing at least one primer, such as an oligonucleotide primer, for amplification of a target nucleic acid (e.g., DNA), wherein the primer is adapted to conform into a structure that can dissociate from a DNA duplex structure in the absence of heating. In other words, and referring to FIG. 2, the primer 20 includes a sequence that naturally conforms into a structure, such as a quadruplex structure (or any other non-B DNA configuration or other DNA structure) in which intramolecular base pairing allows or causes the primer to dissociate from the double stranded DNA—of which it is one strand—and form its particular structure. This structure may be referred to herein as a “dissociative structure” 22 or “dissociative conformation” or the like. As one nonlimiting example, the primer may be adapted to conform into a quadruplex structure. The primer may conform into the quadruplex structure during an extension step of PCR. As this occurs, the primer necessarily separates form its binding site on the target DNA sequence 24 while the extending portion (DNA polymerase adding dNTPs to the sequence) remains bound (at least temporarily) in a double-stranded configuration. Again, it will be recognized by those of skill in the art that quadruplex structures are merely exemplary, and the primer may form any other structure that can disassociate from any DNA configuration of which it is a part.

Further, the primers used in DSPA may be universal. In other words, each primer in the primers used in the reaction includes the same sequence (or a substantially similar sequence that allows amplification to occur). As is known to those of ordinary skill in the art, in standard PCR at least two different primers (i.e., having two different sequences) are used (i.e., a first set of primers, wherein each primer of the first set includes the same or similar sequence, and a second set of primers wherein each primer of the second set includes the same or similar sequence, that sequence being different from the sequence of the primers in the first set). The need for the two sets of primers is to provide for amplification using each of the single strands from the DNA. Thus, one strand will be replicated using primers from the first set. The second strand (which is complementary to the first strand) will also be replicated. However, as that second strand is complementary to the first strand, the primers of the second set may be complementary to the primers of the first set. This creates the problem of primer-dimers (when the primers hybridize to one another—rather than to the target template denatured DNA strands). However, in DSPA, each of the primers used have the same or similar sequence. There is no second set of complementary primers that is needed. As such, there are no other complementary primers for the primers of the present application to hybridize to, thus eliminating the problem of primer dimers. Further, as the end being extended is currently bound with the target region, self-annealing of product is eliminated. And, the original primer binding site on the target DNA region is open for binding of another primer.

While “at least one primer” and a site being “open for binding of another primer” are discussed herein, it will be recognized by those of ordinary skill in the art that the “at least one primer” and the “another primer” may have the same sequence (or substantially similar sequence) as it is known that PCR employs multiple copies of a primer for amplification of a target DNA sequence. Further, as is known to those of ordinary skill in the art, the reaction components generally include multiple copies of primers. Thus, it will be understood that “at least one primer” or “one primer” or “a primer” or “the primer” or like references may refer to a single primer, or a primer among a set of multiple copies of the same or similar primers. Further, while various PCR procedures described herein are discussed as amplifying DNA, those of ordinary skill in the art will recognize that does not limit the disclosure to those seeking DNA sequences, as procedures such as reverse transcription PCR are well known, wherein reverse transcriptase reverse transcribes RNA into cDNA, which is then amplified by PCR.

Further, various nucleic acids, segments thereof, sequences, etc. are referred to herein as being “complementary” to one another. As used herein, “complementary” does not require an exact base-pair matching between each base of complementary sequences, for example. It only requires enough of a match that the sequences are capable of hybridizing to one another.

One aspect of DSPA, then, uses the free energy of dissociative structures (such as quadruplexes) to drive unfavorable (endergonic) reactions of nucleic acids (e.g., isothermal PCR). A key point of these reactions is that some sequences—e.g., some G-rich sequences—are capable of forming structures/conformations with significantly more favorable thermodynamics than the corresponding DNA duplexes. The sequences are incorporated within DNA duplexes, which after interaction with an initiator (e.g., DNA polymerase) self-dissociate from the complementary strand and fold into their dissociative structures (e.g., quadruplexes). The energy of formation of these structures is used to drive PCR at substantially constant temperature.

Because DSPA inhibits product self-annealing and increases the number of PCR cycles within the exponential growth phase, the efficiency of PCR is improved by elongating the window of exponential amplification. And, since the dissociative structure is more stable than its corresponding duplex, unfolding of the duplex or release of target for the coming primers can occur without the need of substantial temperature change or any temperature change. In other words, in standard PCR, following the extension step, the DNA is in a duplex form. The next cycle then begins by raising the temperature to a point that the double-stranded DNA again denatures (i.e., separates into single strands). This is necessary in order to provide the separated sense and antisense DNA strands for primer binding (to each of the strands), followed by elongation during the next extension step (once the temperature of the reaction is reduced). However, by using primers based on the principles of DSPA, the primers and extending nucleotides that are added during the extension step naturally conform into the dissociative structure (such as a quadruplex). As this occurs, the primer (forming the dissociative structure) naturally separates from the target DNA sequence complementary to the primer, thereby leaving the target region complementary to the primer exposed in single-stranded form for binding of the next primer. This occurs without requiring raising of the temperature to denature the strands from one another. Thus, once begun, DSPA can proceed under isothermal conditions. As described above, isothermal DNA amplification is desirable because it would not require expensive instrumentation for thermocycling (as does standard PCR) and thus would allow for DNA amplification in the field and at point-of-care.

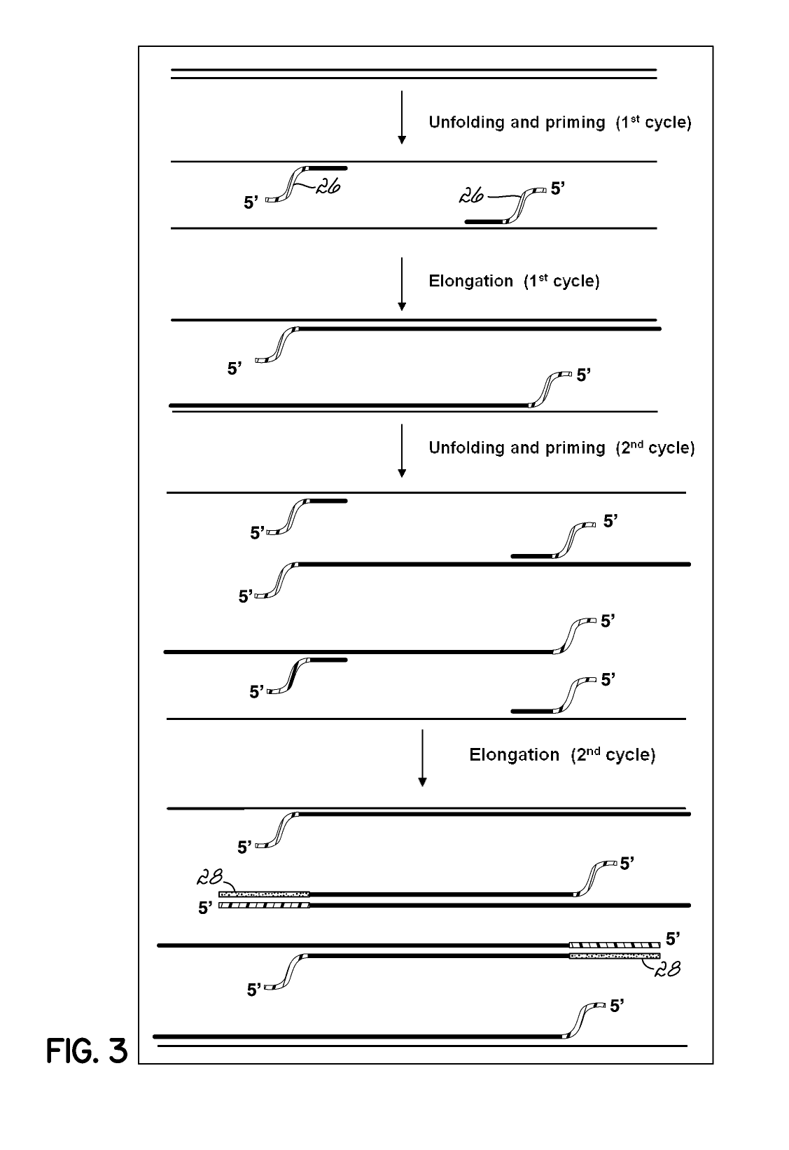

However, as described in the Background, DSPA is not completely isothermal due to the need to incorporate primer binding sites (PBS) into the target DNA, by using cycles of traditional PCR. The incorporation of the target sequence into a template is shown schematically in FIG. 3. The quadruplex folding sequence (hash-marked segment) 26 will be attached at the 5′-end of both forward and reverse primers. The products of the 2nd cycle (four duplexes at the end of PCR, FIG. 3) contain two single-stranded amplicons fully complementary to each other with incorporated target sites at the 3′-end (dotted segments) 28. Thus, at the end of the second cycle, the number of amplicons with incorporated target sites equals the initial amount of template. The use of the initial two cycles of traditional PCR to incorporate PBS prevents this version of DSPA from being completely isothermal. One aspect of the present invention, then, provides a nucleic amplification process that is completely isothermal such that it would be suitable for point-of-care analysis.

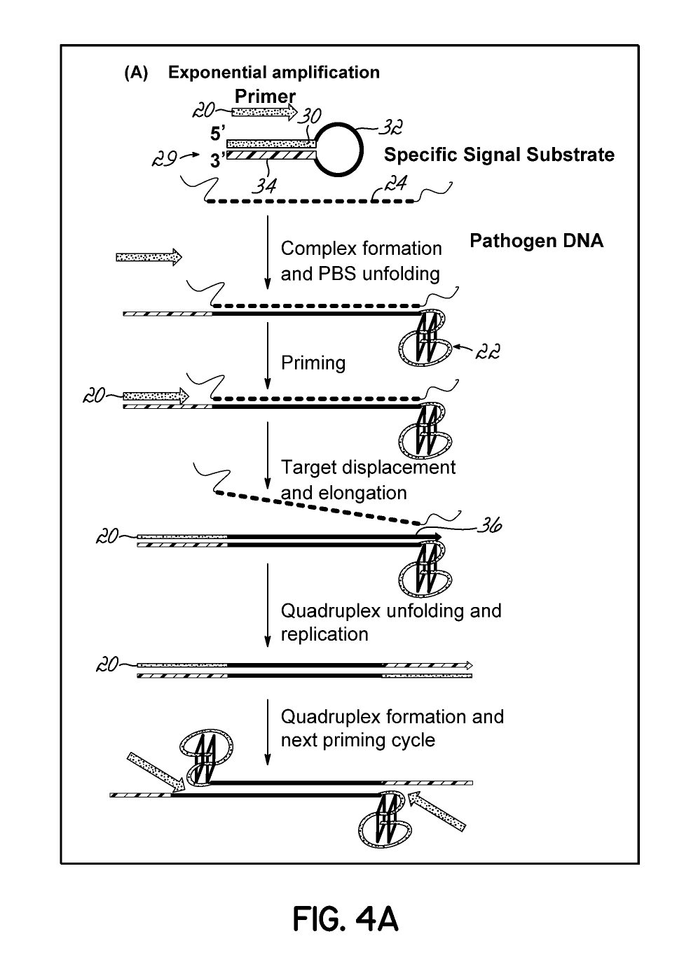

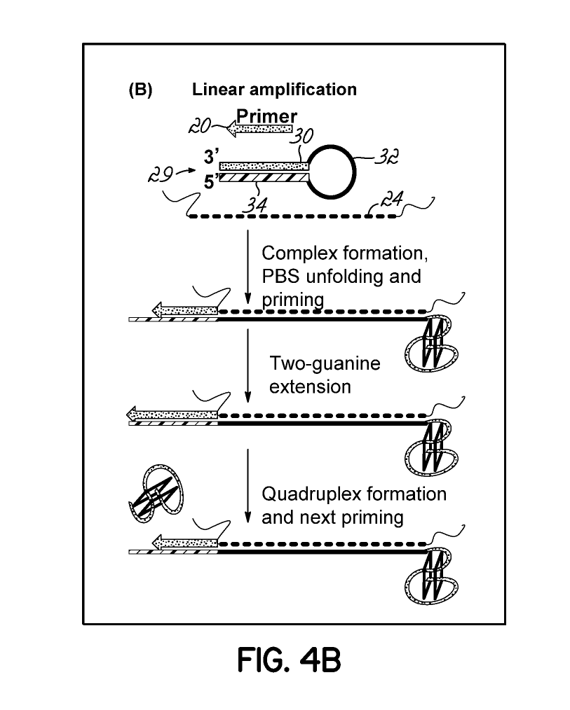

In general, and referring now to FIGS. 4A and 4B, this aspect of the present invention includes at least one nucleic acid construct 29 including first, second and third sequence segments 30, 32, 34 (dotted segment, black line, and hash-marked segments, respectively, in FIGS. 4A and 4B). This construct may be used to identify target nucleic acid 24 (dashed line) via an amplification process wherein it may be the construct itself that is amplified. At least a portion of the first sequence segment 30 includes a sequence adapted to conform into a structure that dissociates from a complementary strand of a DNA duplex (i.e., a dissociative sequence adapted to form a dissociative structure). At least a portion of the second sequence segment 32 includes a sequence that is complementary to a target nucleic acid. And, at least a portion of the third sequence segment 34 includes a sequence that is complementary to the dissociative sequence portion of the first sequence segment. The nucleic acid construct may include a detectable label, so that the presence of the target nucleic acid can be confirmed.

The nucleic acid construct of this aspect of the present invention may have a stem-loop configuration. As is known to those of ordinary skill in the art, stem-loop intramolecular base pairing is a pattern that can occur in single-stranded DNA, or in RNA. The structure may also be referred to as a “hairpin” or “hairpin loop.” It occurs when two regions of the same strand that are generally complementary in nucleotide sequence when read in opposite directions, base-pair to form a double helix that ends in an unpaired loop. In the nucleic acid construct of this aspect of the present invention, at least a portion of the loop region may be complementary to the nucleic acid of interest (i.e., the second sequence segment may be in the loop region). The stem region is then formed by the dissociative-structure-forming sequence (i.e., first sequence segment—30) duplexed with its complementary strand (i.e., third sequence segment—34). In the presence of target nucleic acid 24, the target nucleic acid binds to the loop region 32 of the nucleic acid construct and unfolds the construct, which releases the third sequence segment from the first sequence segment. This frees the third sequence segment (dashed/dotted line) to be bound by primers 20 (short arrow) having a sequence complementary to the third sequence segment, thereby initiating the amplification reaction. In other words, the third sequence segment provides a PBS. Further, the dissociative-structure-forming sequence included in the first sequence segment of the nucleic acid is a sequence such as would be used for a primer in DSPA. Due to the use of sequences that form dissociative structures, the primers and first sequence segments can dissociate from complementary sequences without having to change the temperature of the reaction.

This aspect of the present invention, then, provides an amplification process that is isothermal, which is accomplished, in one aspect, by changing the target that is amplified. For example, current real-time nucleic acid detection mechanisms usually are based on amplification of target nucleic acid followed by quantification. However, an aspect of the present invention provides a detection mechanism that amplifies a nucleic acid construct specific to the target of interest (rather than the target nucleic acid itself) for further quantification. This approach has at least two advantages: (i) it provides a truly isothermal mechanism; and (ii) it can be used in detection of RNA pathogens, such as HIV, without reverse transcription.

As described above, in one aspect, a portion of the first sequence segment may be based on any sequence that is capable of forming a structure that allows or causes that portion to dissociate from a duplex form, such as under isothermic conditions. And, as described above, an example of a structure which allows such dissociation is a quadruplex structure. As previously discussed, such quadruplex structures may be commonly formed by sequences rich in guanine residues. Thus, some of the discussion below is directed to G-rich sequences, which are capable of forming such quadruplex structures. However, it will be appreciated by those of ordinary skill in the art that the general and particular sequences described below, and the particular structures, such as quadruplex structures, described below, are merely exemplary and that there may be other useful sequences that form structures which allow dissociation from a double-stranded DNA form in accordance with the principles of the present invention.

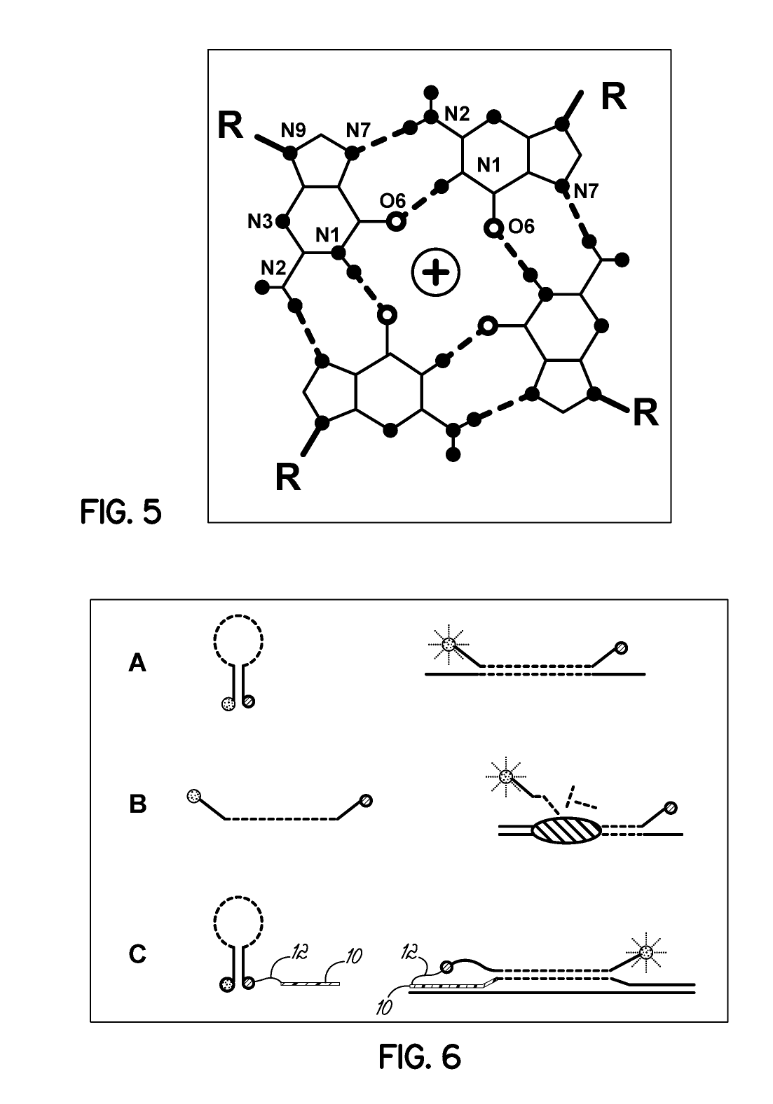

As is known to those of ordinary skill in the art, quadruplexes are high-ordered nucleic acid structures (DNA or RNA) formed from G-rich sequences that are built around tetrads of hydrogen bonded guanine bases (see FIG. 5). Thus, in order to provide a first sequence segment that can form quadruplexes, the first sequence segment of this aspect of the present invention may be designed with a sequence having a G content of a high enough amount (or to obtain a high enough amount) to allow the first sequence segment to conform into a quadruplex structure. In one embodiment, the G content of the sequence of the first sequence segment of this aspect of the present invention is equal to or greater than 70%. In another embodiment, the G content may be equal to or greater than 75%. More specifically, in one embodiment, the first sequence segment may have a sequence based on GGGTGGGTGGGTGGGT [SEQ. ID. NO. 3] [“(GGGT)₄”]. This sequence can form into a quadruplex. However, it will be recognized by those of ordinary skill in the art that the first sequence segment for use in this aspect of the present invention does not have to include the exact (GGGT)₄ sequence. By being “based on” the sequence (GGGT)₄, those of ordinary skill in the art will recognize that substitutions and/or deletions may be made to this base sequence, so long as the resulting first sequence segment based on the (GGGT)₄ sequence remains able to conform into a quadruplex structure. For example, as described above, other sequences may form quadruplexes provided they include a guanine amount that is sufficient to form such quadruplexes. Further, the sequences do not have to be based on (GGGT)₄, as there are other formulas that the sequences may be based on. One example of such a formula is d(G₃+N₍₁₋₇₎G₃+N₍₁₋₇₎G₃+N₍₁₋₇₎+G₃).

Further, and still referring to FIGS. 4A and 4B, this aspect of the present invention provides two types of isothermal amplification: one is an exponential amplification (shown in FIG. 4A), and the other is a linear amplification (shown in FIG. 4B). In both types of amplification, the reaction includes a nucleic acid construct 29 in the form of a stem-loop with a sequence in the loop region 32 that is complementary to the target nucleic acid 24. The stem portion of the nucleic acid construct is formed by a dissociative-structure-forming sequence (e.g., a quadruplex-forming sequence) 30 duplexed with its complementary strand 34. As can be seen in FIG. 4A, the dissociative-structure-forming sequence 30 of the stem is positioned proximal to the 5′ end of the molecule. And in panel FIG. 4B, the dissociative-structure-forming sequence 30 of the stem is positioned proximal to the 3′ end of the molecule. As will be discussed below, these different positions result in exponential amplification versus linear amplification. The complementary strand 34 also provides a primer binding site (PBS) for the primer 20, which is another component of the assay (designated by the arrows in FIGS. 4A and 4B). The primer and stem-loop coexist metastably before adding a target nucleic acid. In other words, in the absence of target nucleic acid, the primer remains free because the primer binding site is duplexed with the dissociative-structure-forming sequence of the stem-loop. Once added to the reaction mixture, the target nucleic acid binds to the loop region of the construct and unfolds it, which releases the PBS for binding with the primer, thereby initiating the amplification reaction (as described above).

When the at least one primer is free in the reaction mixture including at least one nucleic acid construct and at least one target nucleic acid, the primer remains in a form that does not assume a dissociative structure (e.g., it does not spontaneously fold into a quadruplex). As described above, the primer has a similar sequence to the 5′ end of the stem-loop conformation of the nucleic acid construct. And, as described above, that 5′ end of the nucleic acid construct is designed to form into a dissociative structure. However, the primer is formed as a shortened or truncated version of the sequence that appears at the 5′ end of the nucleic acid construct. As a result, the primer will not, on its own, form a dissociative structure. Rather, any extension involving the primer needs to first occur before it can form a dissociative structure (as will be described in greater detail below).

As mentioned above, FIG. 4 depicts two versions of this amplification process. FIG. 4A shows exponential amplification of the nucleic acid construct 29, and FIG. 4B shows linear amplification of the nucleic acid construct 29. One difference between these versions is the location of the first sequence segment (i.e., dissociative-structure-forming sequence) and the third sequence segment (i.e., PBS) in the stem-loop constructs. In FIG. 4A, the dissociative-structure-forming sequences is proximal the 5′ end and the PBS is proximal the 3′ end. In FIG. 4B, the orientation is opposite, with the PBS proximal the 5′ end and the dissociative-structure-forming sequence proximal the 3′ end.

Referring now to FIG. 4A, exponential amplification is as follows: The reaction mixture includes at least one primer(s) 20, at least one nucleic acid construct(s) 29 (e.g., in stem-loop form), and target nucleic acid(s) 24. Target nucleic acid 24 binds to the loop region 32 of the construct 29 and unfolds it, which frees the PBS 34 (i.e., third sequence segment) for binding (with a primer 20), and initiates the amplification reaction. In other words, the dissociative-structure-forming sequence included in the first sequence segment 30 of the nucleic acid construct 29 is a sequence such as would be used for a primer in DSPA. The third sequence segment 34 which is complementary to that first sequence segment 30 therefore has a sequence that can provide a primer binding site for any primer 20 having the sequence of the first sequence segment (or similar sequence).

In order to create the amplification reaction, then, the nucleic acid stem-loop construct 29 described above is placed in a reaction with target nucleic acid 24, and at least one primer 20 having a dissociative-structure-forming sequence similar to, or the same as, the dissociative-structure-forming sequence in the first sequence segment (although these primers are free in solution and are not part of the stem-loop described above). Further, the primers are generally a truncated version of the dissociative sequence portion of the first sequence segment, such that they do not spontaneously form a dissociative structure, such as a quadruplex.

When the target nucleic acid 24 binds to the loop region 32 of the stem-loop, the stem-loop is unfolded. This unfolding releases the third sequence segment 34 (having a PBS) from the first sequence segment 30. The first sequence segment 30 will then spontaneously form its dissociative structure 22 (such as a quadruplex), and the PBS 34 remains free for the primers 20 in the mixture to bind thereto and start an amplification reaction.

As the primer 20 attaches to the PBS 34, during the extension step, then, it amplifies the loop portion of the now-unfolded stem-loop construct. As this occurs, the target nucleic acid 24 will be released from the extending duplex of unfolded stem-loop structure and primer 20/extending sequence 36, thereby allowing the target nucleic acid 24 to be free for binding to another stem-loop nucleic acid construct 29. As extension continues, the extending sequence of nucleotides will confront the sequence of the stem-loop (i.e., the first sequence segment) in the form of its dissociative structure. And upon further extension, the activity of Taq polymerase will convert the first sequence segment back into a duplex. Once Taq subsequently leaves this duplex (after completing assembly of dNTPs in the duplex), the first sequence segment will again dissociate from its complementary strand, uptake K⁺, and form a dissociative structure (e.g., quadruplex). Meanwhile, the primer at the 5′ end of the extending sequence will also assume its dissociative structure. This allows a new primer to attach to the now freed primer binding site (PBS) to continue the amplification process.

Additionally, as shown in the last step of FIG. 4A, Taq must displace the target nucleic acid. However, this will not be a problem since it has already been shown that DSPA works isothermally at 70-75° C., which confirms that at these temperatures Taq unfolds DNA duplexes efficiently.

Referring now to FIG. 4B, a process for linear amplification will be described. As discussed above, the nucleic acid stem-loop construct 29 used in linear amplification, as shown in FIG. 4B, has an opposite configuration as compared to the nucleic acid stem-loop construct 29 of FIG. 4A. Referring to FIG. 4B, one can see that the dissociative-structure-forming portion (of the first sequence segment—30) of the nucleic acid stem-loop construct is located at the 3′ end of the molecule, whereas the PBS 34 is located at the 5′ end of the molecule. This nucleic acid stem-loop construct is combined with primer(s) 20 (arrow) and target nucleic acid 24. Upon combination, the target nucleic acid 24 binds to the loop region 32 of the nucleic acid stem-loop construct 29, thereby unfolding the stem-loop, and unwinding the stem. As this occurs, the PBS 34 is freed from the dissociative-structure-forming sequence (first sequence segment) 30, and the dissociative-structure-forming sequence assumes its dissociative confirmation 22 (e.g., quadruplex). With the PBS 34 now freed, it can hybridize to the primer 20 in the reaction mixture. However, rather than extending the sequence in a direction that causes displacement of the target nucleic acid, extension proceeds in an opposite direction. The sequence of the third sequence segment 34 is chosen so that binding of the primer 20 followed by extension will cause successive guanine residues (e.g., two guanine residues) to be added to the primer sequence. This causes that primer to form its dissociative structure 22 (e.g., quadruplex). Once this structure has formed, the primer binding site is again opened for another primer to hybridize, extend, and the process repeats itself.

As described above, the primers used are of a sequence that does not initially form a dissociative structure (such as a quadruplex), but that will do so upon extension of the sequence during amplification (such as by the addition of guanine residues). One exemplary embodiment of such a primer sequence is GGG(2Ap)GGGTGGGTG (G3T-ss13). Those of ordinary skill in the art will recognize that G3T-ss13 is merely an example of a sequence that can be used as a primer in this aspect of the present inventions, and that other sequences that are capable of forming dissociative structures upon extension may be used. Further, those of skill in the art will recognize that G3T-ss13 has a sequence based on (GGGT)₄. It is a truncated version of (GGGT)₄ and has a detectable label—2Ap (2-aminopurine)—incorporated in the sequence. Those of skill in the art will recognize that other labels may be used in dissociative sequences.

As a result, signal evolves after adding guanines to the primer (as mentioned above and as will be described in greater detail below, a detectable label may be included in the dissociative-structure-forming sequence, which becomes detectable once the dissociative sequence—such as a quadruplex—is formed). Due to the fact that high amounts of dNTP (˜0.5 mM) may inhibit Taq, the reaction shown in FIG. 4B, which only needs, for example, a two-guanine extension in the described embodiment, may allow very high concentrations of signal molecules to form, such that the signal may be detected by the unaided eye.

During the design of nucleic acid constructs of this aspect of the present invention, particular care should be taken to avoid misfolding of the stem-loop, via techniques that are well known to those of ordinary skill in the art. Avoidance of misfolding of the stem-loop will prevent mispriming in the absence of target nucleic acids. Further, while the system shown in FIG. 4B is a linear amplification pattern, the detection process will be accelerated by the fact that it requires only slight elongation (e.g., two-nucleotide elongation), after which the quadruplex (or other conformation) quickly dissociates. Since this mechanism does not require quadruplex replication, the appropriate ionic strength can be achieved by K⁺ ions alone, which will further accelerate signal amplification. Further, one can design several stem-loop molecules complementary to the target nucleic acid at different positions, to further accelerate signal amplification, as will be appreciated by those of ordinary skill in the art.

A problem that may arise during linear amplification, such as that shown in FIG. 4B, is “leakage.” “Leakage,” as referred to herein, is generally a problem of false positives that can occur due to the ability of the 3′ end of the stem loop probe to form a dissociative structure, such as a quadruplex. As has been described above, the stem of the stem loop probe includes (1) a 5′ segment complementary to a primer (the primer having the ability to form a structure such as a quadruplex), and (2) a 3′ segment that is complementary to the 5′ segment. As the 3′ segment is complementary to the 5′ segment, it (like the primer) also has the ability to form a dissociative structure such as a quadruplex. “Leakage” is the problem that occurs when that 3′ end (of the stem loop probe) forms into the quadruplex or other dissociative structure in the absence of any target nucleic acid. This spontaneous formation of the quadruplex then frees the 5′ end of the stem loop probe for binding of the primers (which include 2Ap), which then form quadruplex structures and the 2Ap can be detected. This results in a false positive, since these readings can occur in the absence of target nucleic acid.

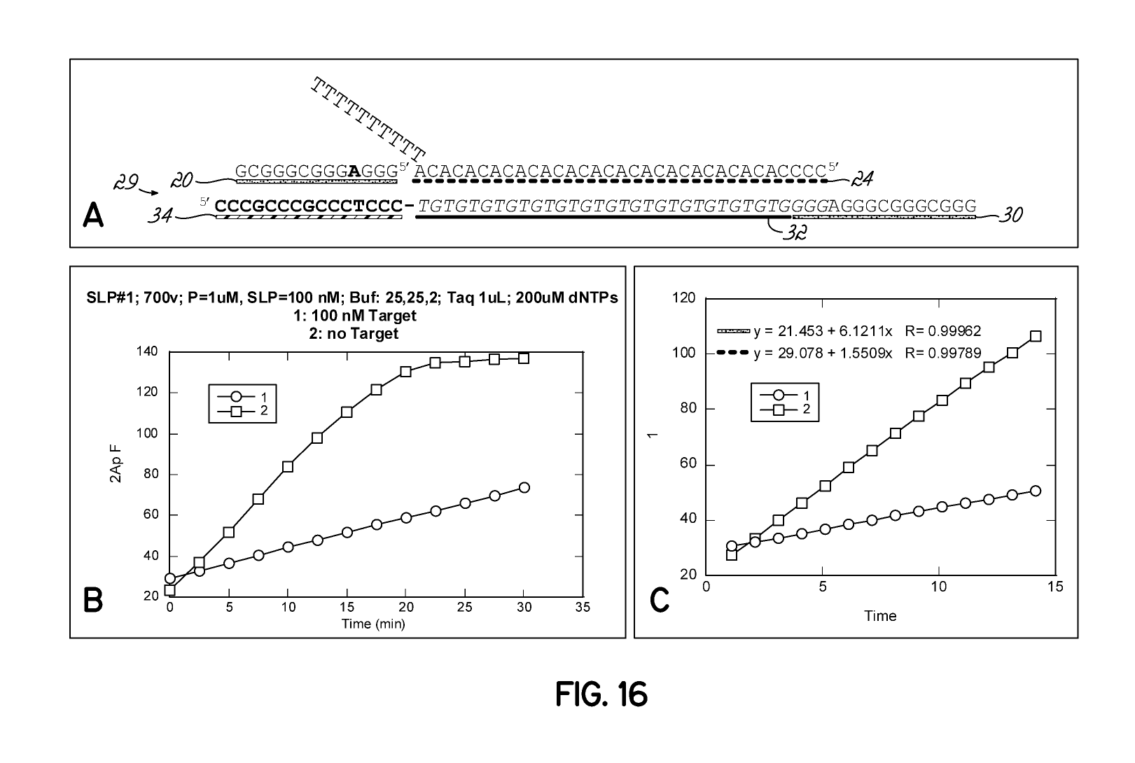

For example, FIG. 16 shows (1) in panel A, a sequence of an exemplary stem loop probe 29 (the 5′ to 3′ sequence at the bottom of panel A, with primer binder site underlined with hash-marked line 34, loop portion underlined 32, and dissociative-structure-forming portion underlined by dotted segment 30) as would be used in linear amplification (as shown in FIG. 4B) unfolded and bound to target nucleic acid 24 (underlined by a dashed line in panel A) and primer 20 (underlined by hash-marked line in panel A); and (2) in panels B and C, graphs demonstrating that the stem loop probe of this embodiment leaks since its 3′ end is able to form a dissociative structure, such as a quadruplex. The exemplary stem loop probe shown in FIG. 16 is 60 nt long, has a 15 by stem, and a 30 nt loop. And the target sequence is 33 bp. As can be seen from the graphs in panels B and C of FIG. 16, the presence of target nucleic acid in a reaction cause rapid primer binding, primer quadruplex formation, and detection of 2Ap (-□-). However, the reaction mixture including no target nucleic acid also results in an increase in primer binding, primer quadruplex formation, and detection of 2Ap (circled line -∘-).

In order to solve this problem of leakage (and false positives), in another aspect of the present invention, the 3′ end of the stem loop probe may include a substitution, such as a G->C substitution, which creates a CC mismatch between 3′ end of probe and 5′ end of target. This substitution prevents the 3′ end of the stem loop probe from forming a quadruplex (or other dissociative structure). While this is described above as a CC mismatch due to a G->C substitution, those of ordinary skill in the art will recognize that other methods may be used to achieve the result. For example, a G may be deleted. And so those of skill in the art will recognize that any sequence which remains complementary to the 5′ end of the stem loop probe, but which will not form a quadruplex or other dissociative structure, will suffice for this aspect of the present invention.

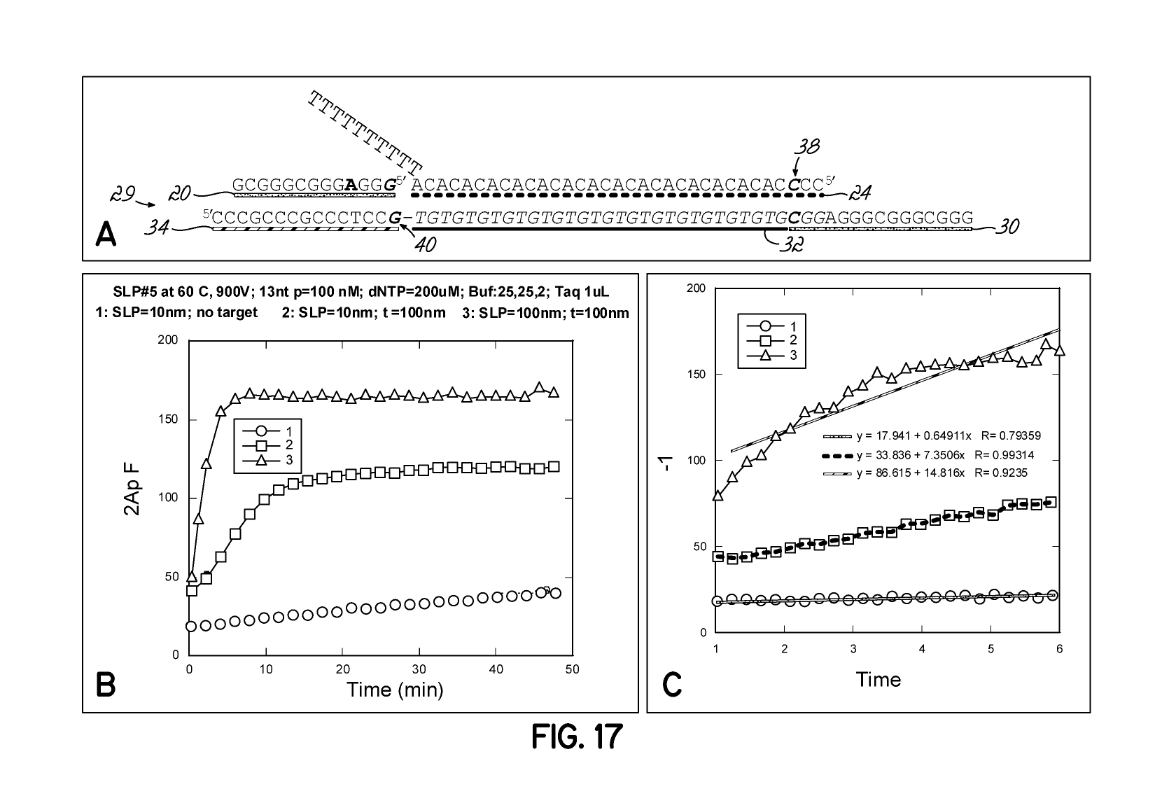

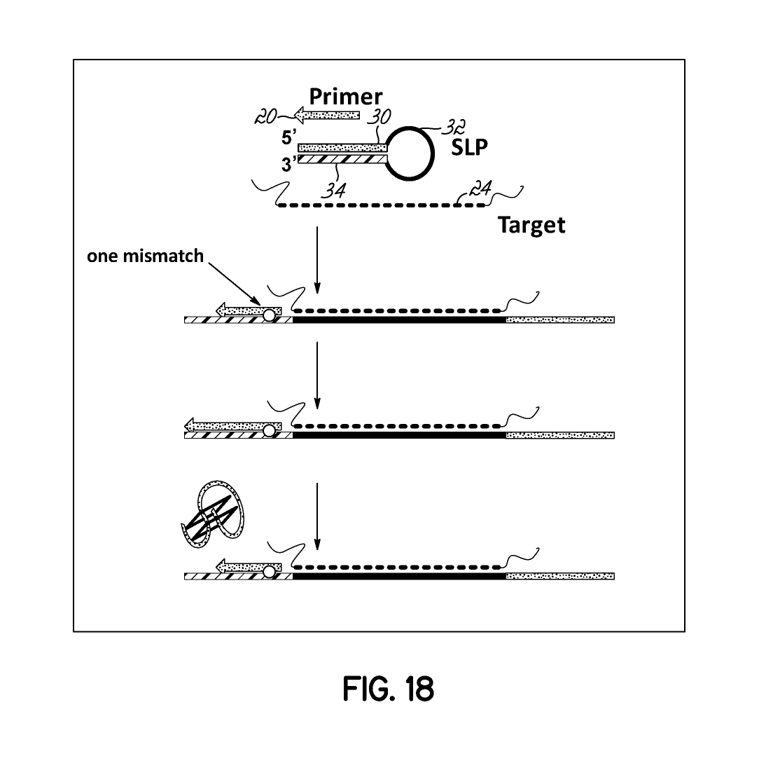

For example, FIG. 17, panel A, shows a sequence of an exemplary stem loop probe including a CG base pair (which previously had been a GC basepair from the version of the stem loop probe in FIG. 16), which does not destabilize the duplexed part of the stem-loop probe, but does cause a CC mismatch 38 between the target nucleic acid 24 and the 3′ end of stem loop probe that inhibits quadruplex (or other dissociative structure) formation. This prevents the 3′ end from forming a dissociative structure (such as a quadruplex). Also, the graphs of FIG. 17 panels B and C demonstrate that leakage (such as shown in FIG. 16) is reduced and eliminated by inhibiting the formation of the dissociative structure. In this exemplary embodiment, the stem loop probe is 60 nt long, has a 15 bp stem, and a 30 nt loop. The target sequence is 33 nt, but the combination of target with stem loop probe is such that there is one CC mismatch (at 5′ end of target/3′ end of probe). (Additionally then, there is a GG mismatch 40 at 5′ end of the probe/3′ end of the primer. However, the primer, incorporating 2Ap (shown by boldfaced “A”) is still able to form dissociative structure and release.) Note that in this exemplary embodiment, the initial slope of the line shown for “no target” (black line) in the graphs of panels B and C is about 11-fold less that the slope of the reaction including target nucleic acid (squared line). Further, as can be seen, the slope of the “no target” line (black line) is much less (closer to zero) than that shown in the graphs of FIG. 16. This demonstrates the result that leakage (such as shown in FIG. 16) is reduced and eliminated by inhibiting quadruplex formation (or other dissociative structure) at the 3′ end of the stem loop probe, such as by the G->C substitution described above. A schematic of this process (as embodied in the example of FIG. 17) is shown in FIG. 18.

As described above, the nucleic acid construct may include a label so that amplification may be detected (e.g., to thereby determine the presence of target nucleic acid). More specifically, in one embodiment, the first sequence segment of the nucleic acid construct and/or the primer may have a label incorporated therein. Such a label may be chosen from labels that are known to those of ordinary skill in the art. Such labels include, but are not limited to, fluorescent labels. And in a particular embodiment, such a label may include 2Ap.

The inclusion of a label in a dissociative sequence overcomes many of the previously described drawbacks of current systems. As described above, quantification methods are based on the fact that the amount of target nucleic acid produced and detected is directly proportional to the initial amount of sample DNA during the exponential growth phase. Since the fluorescence signal during the initial cycles is too weak to be distinguished from the background fluorescence (see the AB segment of FIG. 1) only a narrow window of the exponential growth phase is used for quantification (see the BC segment of FIG. 1). Thus, efficiency would be improved by reducing the background fluorescence (i.e., by the use of well-quenched probes before detection), and by a strong and immediate increase of fluorescence upon amplification, as well as by a longer exponential phase [as described in Edwards, K. J. et al. (2009) In Logan, J. et al. (eds.), Real-time PCR. Caister Academic Press, Norfolk, UK, pp. 85-93; Pfaffl, M. W. et al. (2009) In Logan, J. et al. (eds.), Real-time PCR. Caister Academic Press, Norfolk, UK, pp. 65-83].

As described above currently, four main probes are used to monitor real-time PCR [Lee, M. A., et al. (2009) In Logan, J. et al. (eds.), Real-time PCR. Caister Academic Press, Norfolk, UK, pp. 23-45]: (1) SYBR Green [Becker, A. et al. (1996) A quantitative method of determining initial amounts of DNA by polymerase chain reaction cycle titration using digital imaging and a novel DNA stain. Anal Biochem, 237, 204-207], (2) molecular beacons [Tyagi, S. et al. (1998) Multicolor molecular beacons for allele discrimination. Nature biotechnology, 16, 49-53; Tyagi, S. et al. (1996) Molecular beacons: probes that fluoresce upon hybridization. Nature biotechnology, 14, 303-308], (3) TaqMan® probes [Holland, P. M. et al. (1991) Detection of specific polymerase chain reaction product by utilizing the 5′----3′ exonuclease activity of Thermus aquaticus DNA polymerase. Proc Natl Acad Sci USA, 88, 7276-7280], and (4) Scorpion™ probes [Whitcombe, D. et al. (1999) Detection of PCR products using self-probing amplicons and fluorescence. Nat Biotechnol, 17, 804-807]. And, as described previously, there are drawbacks to each of these. SYBR Green is a dye that intercalates into double-stranded DNA nonspecifically resulting in fluorescence. Although SYBR Green is inexpensive, sensitive and easy to use, it also binds to any double-stranded DNA including nonspecific products or primer dimers.

Referring to FIG. 6, panel A, molecular beacons are single-stranded oligonucleotide probes that form a hairpin-shaped stem-loop structure. The loop contains a probe sequence (dashed line segment, FIG. 6, panel A) that is complementary to a target sequence in the PCR product. The stem is formed by the annealing of complementary sequences that are located on either side of the probe sequence. A fluorophore (dotted circle) and quencher (lined circle) are covalently linked to the ends of the hairpin. Upon hybridization to a target sequence the fluorophore is separated from the quencher and fluorescence increases. Hybridization usually occurs after unfolding of the hairpin and product duplexes in the denaturation step of the next PCR cycle.

As described above, there are several disadvantages with molecular beacons. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences. This introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, molecular beacons can't be used under isothermal conditions. Fifth, design of the probes requires considerable effort and knowledge of nucleic acid thermodynamics. And sixth, probe hybridization involves a bimolecular probe-primer system. This makes the reaction entropically unfavorable, slows down hybridization and complicates product detection at exponential growth. The hybridization is much faster and efficient with monomolecular probe-primer system [as described in Whitcombe, D. et al. (1999) Detection of PCR products using self-probing amplicons and fluorescence. Nat Biotechnol, 17, 804-807].

Referring now to FIG. 6, panel B, TaqMan® probes are single-stranded unstructured oligonucleotides designed to be complementary to a PCR product. They have a fluorophore attached to the 5′ end and a quencher coupled to the 3′ end. When the probes are free in solution, or hybridized to a target the proximity of the fluorophore and quencher molecules quenches the fluorescence. During PCR, when the polymerase replicates a template on which a TaqMan® probe is bound, the 5′-nuclease activity of the polymerase cleaves the probe. Upon cleavage, the fluorophore is released and fluorescence increases. The shortcomings listed above for molecular beacons hold true for TaqMan®. An additional disadvantage of TaqMan® probes is that they require the 5′-nuclease activity of the DNA polymerase used for PCR.

Referring now FIG. 6, panel C, Scorpion™ probes use a single oligonucleotide that consists of a hybridization probe (stem-loop structure similar to molecular beacons) and a primer (10) linked together via a non-amplifiable monomer (12). The hairpin loop contains a specific sequence that is complementary to the extension product of the primer (dashed line). After extension of the primer during the extension step of a PCR cycle, the specific probe sequence is able to hybridize to its complement within the extended portion when the complementary strands are separated during the denaturation step of the subsequent PCR cycle, and fluorescence will thus be increased (in the same manner as molecular beacons). Many of the shortcomings listed for molecular beacons hold true for Scorpion™ probes. First, they require two bulky and costly tags (fluorophore and quencher). Second, the assay requires a separate probe for each template (i.e., mRNA), which dramatically increases the design effort and expense. Third, the mechanism uses separate binding sites for primer and probe sequences. This introduces another component (probe oligonucleotide) to an already complex reaction, and adds additional design limitations due to the need to avoid interactions between the probe and primers. Fourth, hybridization of the probe requires heating steps to unfold the product duplex and hairpin. Consequently, Scorpion™ probes cannot be used under isothermal conditions. And fifth, design of the probes requires considerable effort and knowledge of nucleic acid thermodynamics.

Thus, in certain embodiments, the first sequence segment and/or the primer may include a sequence that is generally based on a sequence in the form of d(G₃₊N₁₋₇G₃₊N₁₋₇G₃₊N₁₋₇G₃) and include a label. In another embodiment, the first sequence segment and/or the primer may include a sequence that is generally based on the (GGGT)₄ sequence and includes a label such as 2Ap. And so, at least a portion of the first sequence segment and/or the primer may have a sequence based on 2Ap-G3T (GGG2ApGGGTGGGTGGG). However, it will be recognized by those of ordinary skill in the art that this sequence is not necessarily the entire sequence of the first sequence segment and/or the primer, merely that the first sequence segment and/or the primer may include the sequence based on 2Ap-G3T as a portion of the overall sequence of the first sequence segment and/or the primer.

In particular, in the illustration of FIG. 2, a primer that is a truncated version of (GGGT)₄ (a 13b primer in the illustrated embodiment) and incorporates 2Ap is used. In another particular embodiment, this primer has the sequence GGG(2Ap)GGGTGGGTGGG (2Ap-G3T—a.k.a. G3T-ss15). When this primer is not in the quadruplex conformation, fluorescence of 2Ap is quenched. In alternate embodiments, the primer may include different, albeit similar, sequences. For example, in one alternate embodiment, the primer may have the sequence GGG(2Ap)GGGTGGGTGG(G3T-ss14). And in another alternate embodiment, the primer may have the sequence GGG(2Ap)GGGTGGGTG (G3T-ss13—as in the illustrated embodiment). As can be seen in the top panel of FIG. 2, before elongation, the primers (here shown as a 13b primer e.g., GGG(2Ap)GGGTGGGTG) form duplexes with the target sequence since they are missing a few guanine residues that would result in quadruplex formation. Elongation then begins, with the DNA polymerase adding dNTPs to the end of the primers (as shown in the second panel of FIG. 2). This elongation then eventually adds the length and/or guanine residues necessary to allow a quadruplex structure to be formed. Once this occurs (see the third panel of FIG. 2), the 5′-end of each product DNA is trapped in a quadruplex and its complementary sequence (the target DNA) is fully accessible to another incoming primer. And with the formation of the quadruplex, 2Ap is no longer quenched. In still further embodiments, the primers may have the sequence GG(2Ap)TGGTGTGGTTGG or may have the sequence GGTTGG(2Ap)GTGGTTGG.

Further, since the conformation taken on by the first sequence segment and/or the primer sequence (such as a quadruplex) is more stable than its corresponding duplex, unfolding of the duplex or release of target for the incoming primers can occur without the need of substantial temperature change or any temperature change. In other words, in standard PCR, following the extension step, the DNA is in a duplex form. The next cycle then begins by raising the temperature to a point that the double-stranded DNA again denatures (i.e., separates into single strands). This is necessary in order to provide the separated sense and antisense single-stranded DNA strands for primer binding (to each of the strands), followed by elongation during the next extension step (once the temperature of the reaction is reduced). However, by using first sequence segments and/or primers based on a dissociative-structure-forming sequence, such as the (GGGT)₄ sequence, the primers plus extending nucleotides that are added during the extension step, and the first sequence segments, naturally conform into a structure such as a quadruplex. As this occurs, the primer (e.g., forming the quadruplex structure) naturally separates from the target DNA sequence complementary to the primer, thereby leaving the target region complementary to the primer exposed in single-stranded form for binding of the next primer (as can be seen in FIG. 4A, this occurs in both strands). This occurs without requiring raising of the temperature to denature the strands from one another. Thus, amplification can proceed under isothermal conditions. And so, the isothermal DNA amplification provided by the present invention does not require expensive instrumentation for thermocycling and may allow DNA amplification in the field and at point-of-care. And, the product yield in this isothermal system may be characterized using real-time fluorescence measurements of 2Ap incorporated within DSPA primers.

Another aspect of the present invention provides protein-free dissociative-structure-based amplification.

As described in the Background, PCR, which depends on the enzymatic activity of DNA polymerases, is not ideally suited for point-of-care use. Polymerization-based amplification yields a macroscopically observable polymer, visible to the unaided eye [Hansen, R. R., Johnson, L. M. and Bowman, C. N. (2009) Visual, base-specific detection of nucleic acid hybridization using polymerization-based amplification. Analytical biochemistry, 386, 285-287]. However, it still depends on DNA polymerization for coupling biotinylated dNTPs to DNA hybrids, and requires 0.5 nM or higher target concentrations. Surface plasmon resonance coupled with interferometry [Kim, D. K., Kerman, K., Saito, M., Sathuluri, R. R., Endo, T., Yamamura, S., Kwon, Y. S. and Tamiya, E. (2007) Label-free DNA biosensor based on localized surface plasmon resonance coupled with interferometry. Analytical chemistry, 79, 1855-1864] detects 10 pM target but requires instrumentation unsuitable for POC diagnostics. Gold nanoparticles can be visualized with the unaided eye at high pM to nM target concentrations [Thaxton, C. S., Georganopoulou, D. G. and Mirkin, C. A. (2006) Gold nanoparticle probes for the detection of nucleic acid targets. Clinica chimica acta; international journal of clinical chemistry, 363, 120-126]. However, to increase sensitivity further, they must be coupled with PCR or other specialized detection platforms. A sandwich-type binding assay is able to detect 60 fmol target DNA, however it depends on biotinylated capture oligonucleotides, repeated washing steps and additional liposome components [Zimmerman, L. B., Lee, K. D. and Meyerhoff, M. E. (2010) Visual detection of single-stranded target DNA using pyrroloquinoline-quinone-loaded liposomes as a tracer. Analytical biochemistry, 401, 182-187]. Hybridization chain reaction [Yin, P., Choi, H. M., Calvert, C. R. and Pierce, N. A. (2008) Programming biomolecular self-assembly pathways. Nature, 451, 318-322] and entropy-driven signal amplification [Zhang, D. Y., Turberfield, A. J., Yurke, B. and Winfree, E. (2007) Engineering entropy-driven reactions and networks catalyzed by DNA. Science (New York, N.Y, 318, 1121-1125] do not require the use of specific detection platforms, and are based on autocatalytic reactions between DNA oligonucleotides in solution. However, both methods use complicated reactions and detection mechanisms. In addition, these methods suffer from low sensitivity due to significant levels of spontaneous autocatalysis even in the absence of target molecules.

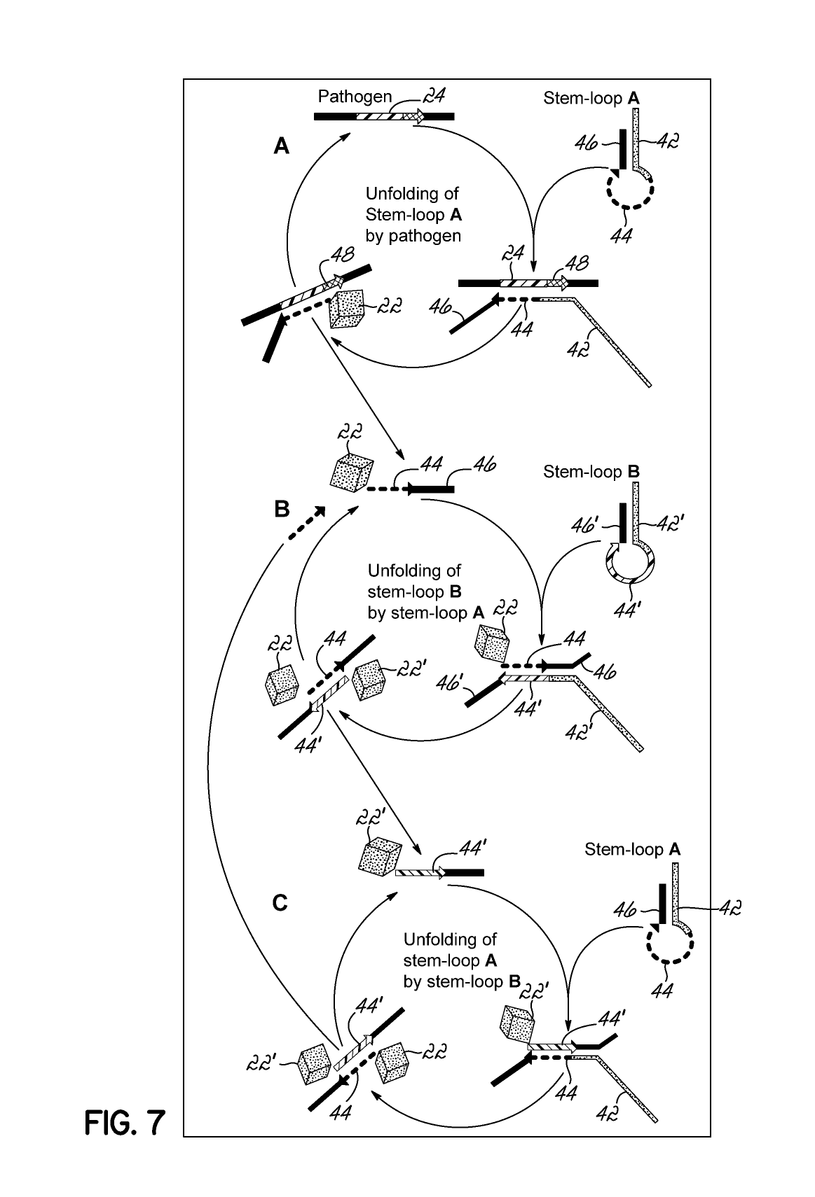

In general, and referring now to FIG. 7, another aspect of the present invention overcomes these drawbacks. This aspect provides a mixture of nucleic acid constructs including at least one first nucleic acid construct and at least one second nucleic acid construct. The at least one first nucleic acid construct and at least one second nucleic acid construct are designed such that they work in concert to provide an amplification reaction that can identify a target nucleic acid sequence (e.g., a DNA sequence) without the use of enzymes as in standard PCR. The reaction in this aspect of the present invention may also proceed isothermally.

To that end, the first nucleic acid construct (designated “stem-loop A” in FIG. 7) includes a first sequence segment 42 (dotted segment) and a second sequence segment 44 (dashed line), wherein at least a portion of the first sequence segment includes a sequence adapted to conform into a structure 22 that dissociates from a complementary strand of a DNA duplex, and wherein at least a portion of the second sequence segment includes a sequence that is complementary to a target nucleic acid sequence 24. And, the second nucleic acid construct (designated “stem-loop B” in FIG. 7) includes a first sequence segment 42′ (dotted segment) and a second sequence segment 44′, wherein at least a portion of the first sequence segment includes a sequence adapted to conform into a structure 22′ that dissociates from a complementary strand of a DNA duplex, and wherein at least a portion of the second sequence segment includes a sequence that is substantially similar to the target nucleic acid such that the second sequence segment 44′ of the second nucleic acid construct (“stem-loop B”) can bind with the second sequence segment 44 of the first nucleic acid construct (“stem-loop A”).

Like the nucleic acid construct for isothermal amplification and identification described above, each of the first and second nucleic acid constructs in this aspect of the present invention may be provided in the form of stem-loop constructs. In each of the stem-loop constructs, the portion of the sequence which includes a dissociative-structure-forming sequence provides a segment of the stem (being duplexed with a complementary sequence 46, 46′—black). A primary portion of the loop of the first nucleic acid construct includes a sequence that is complementary to the target nucleic acid, and a primary portion of the loop of the second nucleic acid construct includes a sequence that is substantially the same as the target nucleic acid sequence.

When the first and second nucleic acid constructs of this aspect of the present invention are combined with target nucleic acid, the target nucleic acid hybridizes with the loop segment of the first nucleic acid construct. This unfolds the stem-loop of the first nucleic acid construct, thereby unwinding the stem. Once unwound, the sequence of the now free dissociative-structure-forming sequence (i.e., the first sequence segment) forms its dissociative structure (hash-marked box—e.g., a quadruplex). As a result, the DNA duplex between the loop segment and the target nucleic acid is destabilized and the complex quickly dissociates. The released target binds to another first nucleic acid stem-loop construct and repeats the same cycle.

Meanwhile, the denatured first nucleic acid construct, now having a dissociative structure 22 at its 5′ end (dotted box), binds to the stem-loop of the second nucleic acid construct [with hybridization between the second sequence segment 44 of the first nucleic acid construct and the loop segment (second sequence segment 44′) of the second nucleic acid construct]. This induces a similar unwinding/dissociation process in the second nucleic acid construct. Once unwound, the first sequence section of the second nucleic acid construct forms its dissociative structure 22′ (dotted box—e.g., a quadruplex). As a result, the DNA duplex between the first and second nucleic acid constructs is destabilized, and the two separate. The released second nucleic acid construct now binds to and unfolds stem-loop of another first nucleic acid construct. At this point, the reaction becomes autocatalytic, i.e., the product of each cycle serves as the catalyst for the subsequent cycles. Thus, amplification occurs in the absence of any standard DNA polymerases, and can proceed isothermally.

A key feature of the system is that the large potential energy of quadruplex formation is captured in DNA duplexes with significantly lower free energies, which is achieved by pre-forming the duplexes in the presence of Cs⁺ and adding quadruplex-forming K⁺ afterward. Thus, after adding K⁺, ions quadruplex formation becomes thermodynamically favorable but kinetically trapped. In other words, K⁺ ions bring the stem-loops to a metastable condition similar to a chain of dominos in the upright position. Adding the target nucleic acid results in the exponential domino effect.

Initially, the target nucleic acid 24 hybridizes with the first nucleic acid construct (stem-loop A) and unwinds the stem (as shown in FIG. 7, panel A). Target nucleic acid 24 hybridizes with the second sequence segment 44 and a few guanines from the first sequence segment 42 and therefore the target nucleic acid 24 should contain a few cytidines at the 3′-end 48 (cross-hatched arrow). This can be accomplished by adding cytidines, where necessary, to the sequence of target DNA. While this may be useful in a laboratory setting, it is not as useful in point-of-care analysis. And so, alternatively, a target segment may be chosen that already has the necessary cytidines (repeating cytidines are common in nucleic acid sequences, as is known to those of ordinary skill in the art). Thus, the newly formed hybrid includes terminal guanines of the dissociative-structure-forming sequence, which is not enough to prevent formation of the dissociative structure (e.g., quadruplex) at the reaction temperature. As a result, the DNA duplex is destabilized and the complex quickly dissociates. The dissociative structure 22 (e.g., quadruplex) formation is accompanied by contraction of the loop segment by a few terminal guanines, which inhibits the reverse reaction between newly dissociated strands. Released target binds to another stem-loop A (see FIG. 7, panel A) and repeats the same cycle, while denatured A binds to the stem-loop B and induces a similar unwinding/dissociation process (see FIG. 7, panel B), which is followed by unfolding of stem-loop A by denatured B (see FIG. 7, panel C). At this point, the reaction becomes autocatalytic, i.e., the product of each cycle serves as the catalyst for the subsequent cycles.

In order to design a successful non-enzymatic signal amplification system, the length of the nucleic acid constructs and experimental conditions should be selected carefully and reactions should be conducted at the appropriate temperature such that: (i) the stem-loop constructs are folded; (ii) the target should be able to bind and unfold the stem-loop; (iii) dissociative structure formation at the end of the stem-loop should be favorable; and (iv) the bimolecular complex between target and hairpin should dissociate itself.

Further, as described above, a drawback of current RT-PCR-specific quantification systems is that they use FRET-based applications (Förster Resonance Energy Transfer), which require costly synthesis and considerable effort to design a sensitive probe. As is known to those of ordinary skill in the art, FRET is a mechanism describing energy transfer between two chromophores. A donor chromophore, initially in its electronic excited state, may transfer energy to an acceptor chromophore (in proximity, typically less than 10 nm) through nonradiative dipole-dipole coupling. Unfortunately, the processes currently used require multiple probes for multiple targets (i.e., one probe for each target), which greatly increases materials, time, and expense.

Another disadvantage of the current RT-PCR detection mechanisms is that two separate functions, recognition and detection, are combined within a probe (see the discussion of currently used process in the Background). The bifunctional nature of the probes requires that the fluorophore-quencher pair be attached to each DNA probe sequence, which makes quantification impractical when several targets are tested. As shown in FIGS. 3 and 4, DSPA separates these two functions, which allows using the same reporter molecule for different targets.

More specifically, DSPA described in FIG. 4 consist of the following steps: (i) template recognition by a stem-loop probe, which is accompanied by release of the PBS; (ii) priming; and (iii) primer elongation, which is accompanied by light emission. By comparison, traditional RT-PCR consists of: (i) template recognition by primers; (ii) amplification; and (iii) recognition of amplicons by a probe, which is accompanied by fluorescence reporting. Thus, in traditional RT-PCR recognition happens twice, while in DSPA it only occurs once. Therefore, to perform 96-well quantification using molecular beacons, for instance, it is necessary that the same fluorophore-quencher pair be attached to 96 different probes, while in DSPA one can use a single primer (e.g., GCGC-G3T-ss17) for all 96 targets. Thus, DSPA uses intrinsic fluorescence of primers and quantifies different templates with the same probe.

Multiplexing, or detecting more than one target in the same tube, requires several primers with (i) similar thermal stabilities, but with high selectivity to their matching binding sites, and (ii) distinct fluorescence properties for each probe. Since DSPA primers are limited to specific guanine-rich sequences and there are only a limited number of intrinsically fluorescent nucleotide analogs, the ability of DSPA to be applied to multiplexing is not obvious.

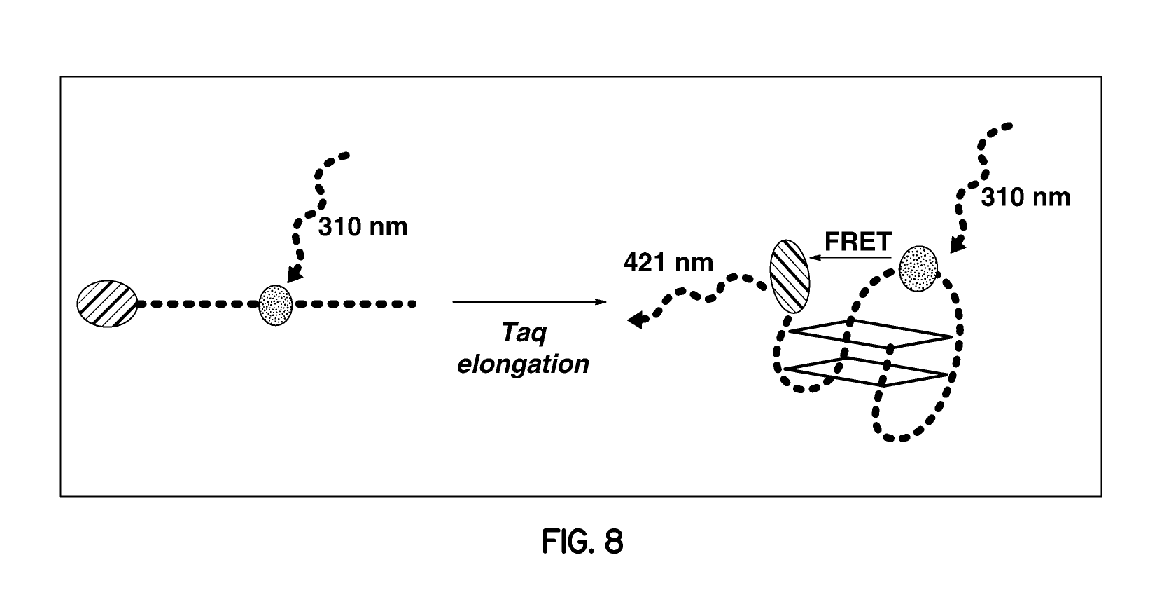

Thus, another aspect of the present invention provides FRET-based DSPA detection, which increases the multiplex capability of DSPA. A fluorescent nucleotide donor will be placed internally and a fluorescent acceptor will be attached at 5′-end of a DSPA primer (FIG. 8). The fluorescent acceptor may be positioned proximal to the 5′ end of the primer. The fluorescent nucleotide donor may be 2Ap. And the fluorescent acceptor may be Alexa405. The fluorescence emission peak of 2Ap overlaps the excitation peak of attached Alexa405. Such a double labeled DNA is commercially available from TriLink Biotechnologies. No fluorescence signal will be observed before quadruplex formation since 2Ap is quenched by adjacent nucleotides. Upon quadruplex formation 2Ap emits light at 370 nm and energy from 2Ap is transferred to Alexa405 resulting in an emission signal at 420 nm. The attachment will increase cost of the synthesis, but since one particular DSPA primer can be used to detect different nucleic acid targets, the overall cost will still be significantly lower than current RT-PCR approach (including multiple probes for multiple targets). Additional 2Ap-based FRET probes may include using Alexa350 (Ex343, Em442) as 5′-end attachment, while pteridine will be coupled with Alexa430 (Ex434, Em541) or Alexa488 (Ex495, Em519). Other fluorescent nucleotides may include pteridine analogs: 3-methyl isoxanthopterin (3MI) (Ex348, Em431), 6-methylisoxanthopterin (6MI) (Ex340, Em430) and (4-amino-6-methyl-8-(2¢-deoxy-â-D-ribofuranosyl)-7(8H)-pteridone (6AMP) (Ex330, Em435).

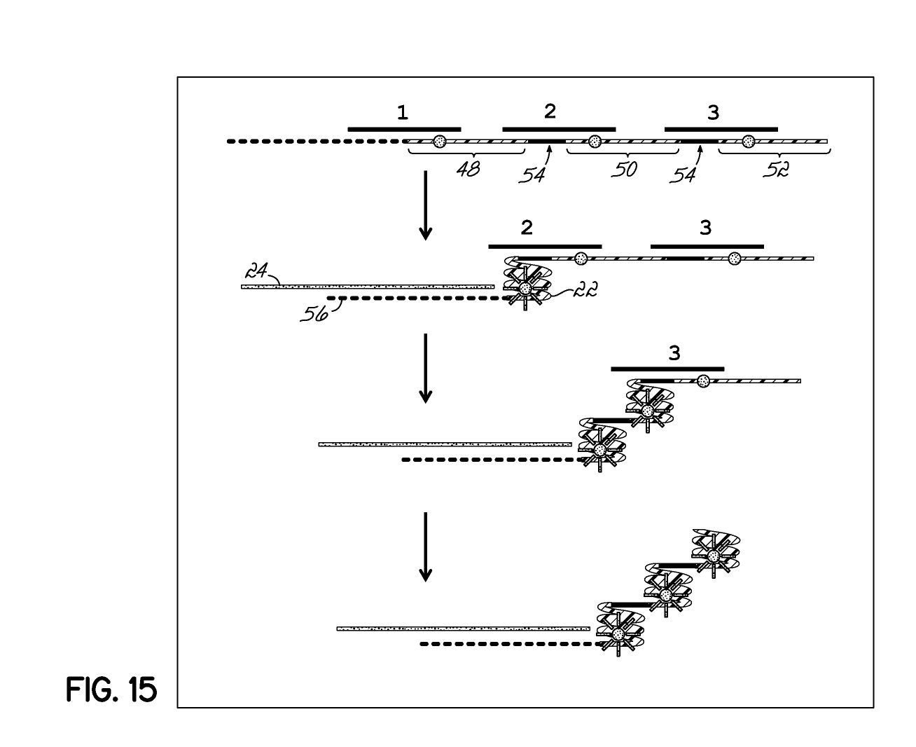

In another aspect of the invention, and referring now to FIG. 15, a nucleic acid construct may be provided that including multiple sequence segments, each having a detectable label, that allows for amplification of the signal generated.

More specifically, the nucleic acid construct may include (1) a first sequence strand, and (2) plurality of nucleotide segments, wherein the plurality of nucleotide segments each include a sequence that is complementary to at least a portion of the sequence of the first sequence strand. As a result, the plurality of nucleotide segments can act as a segmented version of a complementary strand, and, at least initially, retain the first sequence strand in a pseudo-duplex form (as shown in the top panel of FIG. 15).

The first sequence strand of nucleotides includes from the 5′ to the 3′ end: (1) a first segment having a sequence of nucleotides complementary to a target nucleic acid, and (2) a plurality of segments, each of said plurality of segments having a detectable label. Each of the plurality of segments is adapted to conform into a conformation having a free energy with more favorable thermodynamics than a corresponding B-DNA duplex. For example, each of the plurality of segments may be adapted to conform into a quadruplex. Such formation of non-B-DNA duplex structures and their use in accordance with the principles of the present invention is discussed at length above.

As described above, the plurality of segments (numbered 1, 2, and 3 in the first panel of FIG. 15) initially retains the first sequence strand in a pseudo-duplex form. This is accomplished because each of the plurality of nucleotide segments includes a sequence that is complementary to either (1) a sequence spanning the first segment and one of the plurality of segments of the first sequence strand, or (2) at least two of the plurality of segments of the first sequence strand. As can be seen from FIG. 15 (and particularly the top panel thereof), the segment numbered “1” includes a portion complementary to a portion of the first sequence strand that is complementary to target DNA, and a portion complementary to a portion of the first labeled sequence. The segment numbered “2” includes a portion complementary to a portion of the first labeled sequence, and a portion complementary to a portion of the second labeled sequence. And, the segment numbered “3” includes a portion complementary to a portion of the second labeled sequence, and a portion complementary to a portion of the third labeled sequence.

Thus, in operation (and still referring to FIG. 15), the construct in this illustrated embodiment may include three segments 48, 50, 52 having a labeled sequence capable of conforming into a non-B-DNA duplex structure. It will be recognized by those of skill in the art that any number of such segments is possible. In a particular embodiment, the segments 48, 50, 52 may include a sequence such as GGGNGGGNGGGNGGG (where “N” represents fluorescence nucleotides such as 2Ap or 6MI). Such a sequence is merely exemplary as other sequences may be used. Further it is not necessary that each of the segments include the same sequence.

The segments 48, 50, 52 may be connected to each other with a few nucleotides (Ts or Cs) 54 hybridized to three (or more) short segments (separate black segments 1, 2, 3) as shown in the illustrated embodiment. This prevents quadruplex (or other non-B-DNA duplex) formation before hybridization with target nucleic acid. When target nucleic acid 24 hybridizes with its complementary part 56, segment 1 is displaced. This is followed by first quadruplex 22 (or other non-B-DNA duplex) formation, which in turn destabilizes next bimolecular duplex (at segment 2) and so on (i.e., at segment 3, and any other further segments). As a result one can have many signals per construct. In alternate embodiments, one end of the construct (e.g., the left end as shown in FIG. 15) can be attached to a solid surface in a DNA chip, which would allow massive multiplexing.

DSPA results in higher specificity. To produce a false signal, non-specific priming alone is not enough. The non-specifically bound primer would have to bind at cytidine-tracts, which further decreases the possibility of a false signal. Since linear DSPA requires only dGTP and exponential DSPA can be performed in the presence of dGTP and dCTP, non-specific replication could be inhibited by using an incomplete set of dNTPs.

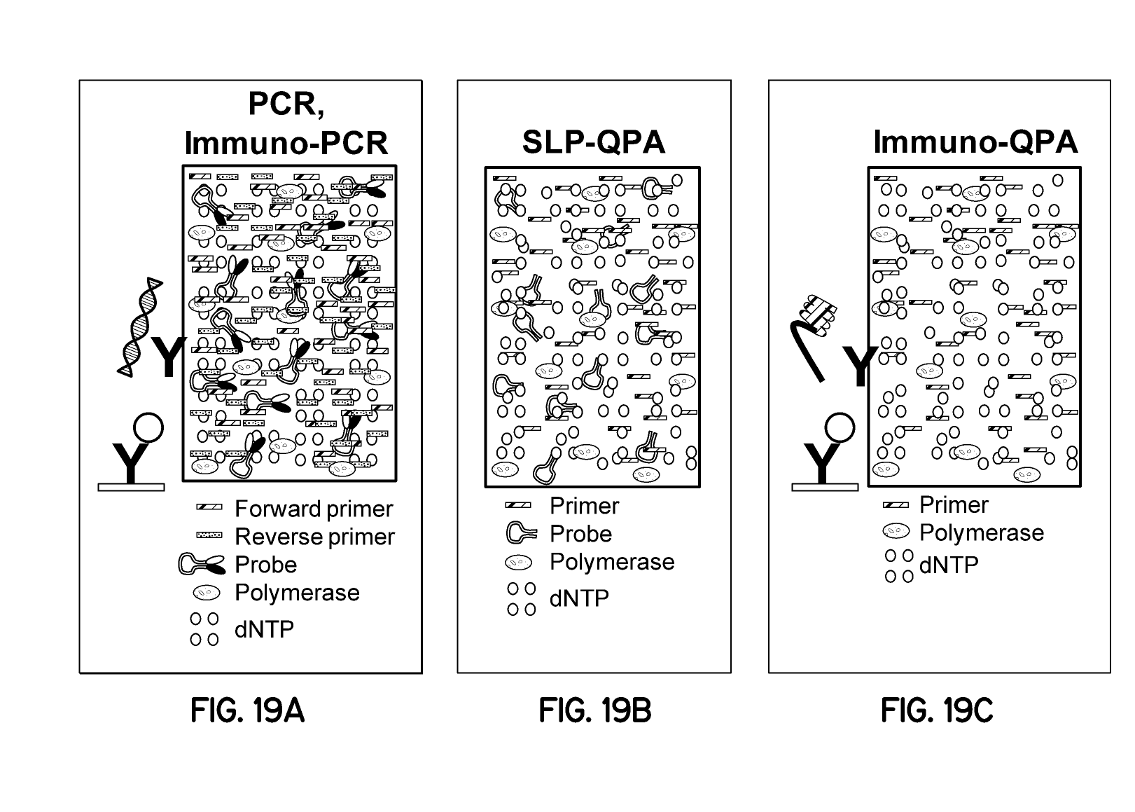

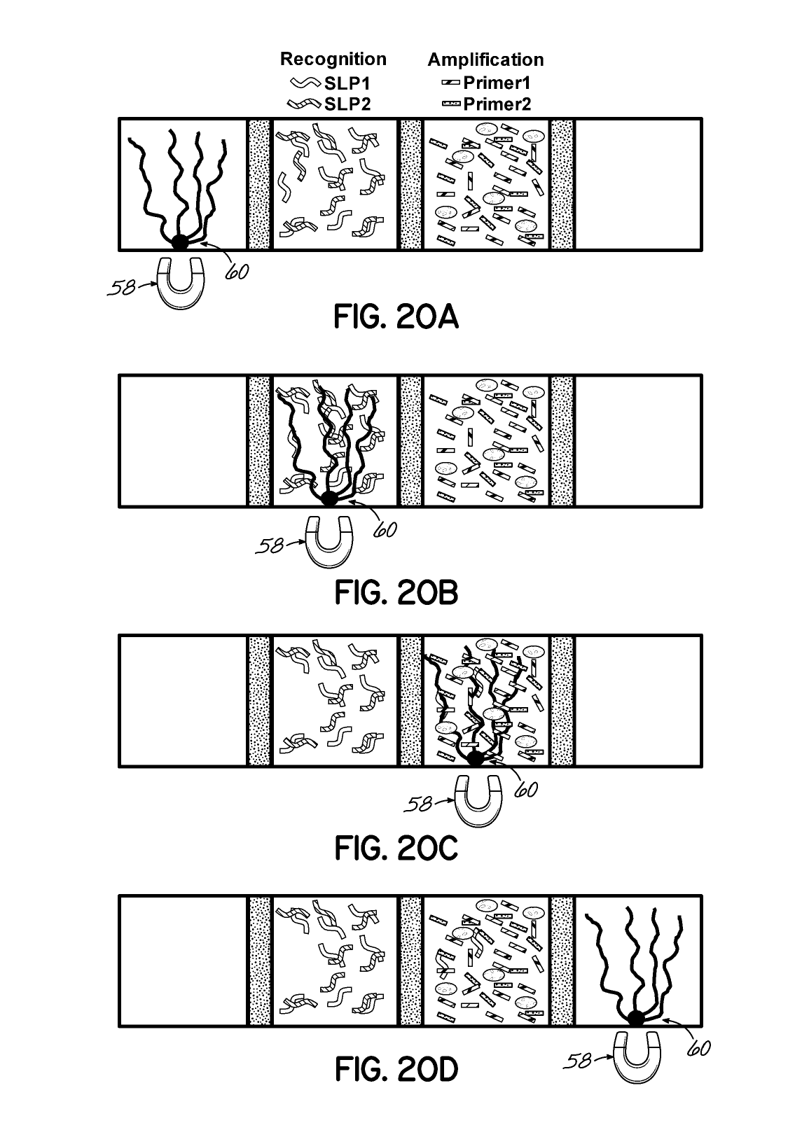

Referring now to FIGS. 19A-19C, one particular advantage of DSPA is shown: DSPA results in a simplified reaction mix as compared to traditional PCR. It also results in a simplified reaction mixture as compared to immune-PCR (an antigen detection system using PCR in which a specific DNA molecule is used as the marker—as described in, for example, Sano et al., Immuno-PCR: a very sensitive antigen detection by means of specific antibody-DNA conjugates, Science, 258 (5079), Oct. 2, 1992, pp. 120-122, incorporated by reference herein in its entirety). Referring particularly to FIG. 19A, one can see a typical PCR reaction as including forward primers, reverse primers, probes, polymerases, and dNTP's. Referring to FIG. 19B, one can see that the reaction mixture for DSPA including stem loop probes includes a single primer, the stem loop probe, polymerase, and dNTP's. And finally, referring to FIG. 19C, immuno-DSPA provides a reaction mixture only including primers, polymerases, and dNTP's. (One of ordinary skill in the art will note that FIGS. 19B and 19C refer to “QPA”—a.k.a. quadruplex priming amplification; however, as discussed above, the primers do not necessarily need to form into quadruplexes, as will be appreciated by those of ordinary skill in the art, but only need to form into any structure that dissociates from a complementary sequence, i.e., DSPA.)

Another advantage of DSPA is shown in FIGS. 20A-D. As described previously, one of the major disadvantages of current RT-PCR detection mechanisms is that two separate functions, recognition and signal production, are combined within a probe. This requires the presence of primers and probes in the same solution, which complicates the reaction (as is shown in FIG. 19A, discussed above). In DSPA, however, these two functions are separated, which allows one to provide recognition and signal amplification in different solutions (see FIGS. 20A-D). As a result, the reactions are less complicated.

Further, in another embodiment of the present invention, a magnetic force or magnetic field 58 may be used to move any target nucleic acid through the solution (or sequentially through different reaction solutions—or steps of a reaction), by having nucleic acid adsorbed onto the surface of metal beads 60. One such type of bead is GeneCatcher™ Magnetic Beads (commercially available from Invitrogen, Carlsbad, Calif.). This allows for a further simplified yet effective reaction that lends itself to use at point-of-care. As has been described above, one benefit of DSPA is that it allows for nucleic acid-based detection at point-of-care or in settings where little resources are available. Current nucleic acid-based detection systems, such as quantitative PCR, are attractive technologies because of their sensitivity and specificity. However, the effectiveness of a typical PCR reaction is dependent upon the quality and quantity of the nucleic acid template, and the absence of interferents (e.g., carbohydrates, proteins, and lipids—which have all been shown to inhibit PCR and product false negatives). To minimize these false negatives and thereby maximize the efficiency of nucleic acid-based diagnostics, nucleic acids are often extracted and concentrated into an interferent-free buffer prior to testing. The methods used to do this are highly effective, but are time-consuming and often require the use of toxic organic chemicals. Other solid phase extraction kits are commercially available to purify DNA or RNA from patient samples, however, many of these kits rely on selective nucleic acid binding to silicone-coated surfaces in the presence of materials such as ethanol and guanidinium thiocyanate. Such kits are not cost-effective for low resource use and often require the use of specialized laboratory equipment and trained technicians, which decrease the effectiveness of their use as point-of-care technologies.

Thus, one embodiment of the present invention may include a reaction vessel including one or more defined sections, with a particular reaction mixture or part of a reaction mixture (e.g., including one or more components of a reaction mixture—primers, etc.) in different sections of the vessel. For example, referring to FIGS. 20A-D, a reaction vessel (such as a cassette) may include a chamber including a solution having stem loop primers (i.e., the second panel of FIGS. 20A-D) and a section including a solution having amplification primers (see the third panel of FIGS. 20A-D). With any target nucleic acid associated with metallic beads, (such as by being adsorbed onto the surface thereof—see the left-most panel of FIG. 20A), a magnetic field may then be moved along the cassette in order to move the metal bead and thus the nucleic acid target sequentially through the various solutions (see the magnet, representing magnetic field moving from panel to panel in FIGS. 20A-D). The general use of such a magnetic field to move metal beads with adsorbed nucleic acid through such cassettes is described in Bordelon et al., Development of a Low Resource RNA Extraction Cassette based on Surface Tension Valves, Applied Materials and Interfaces, 2011, 3, 2161-2168, which is incorporated by reference herein in its entirety.

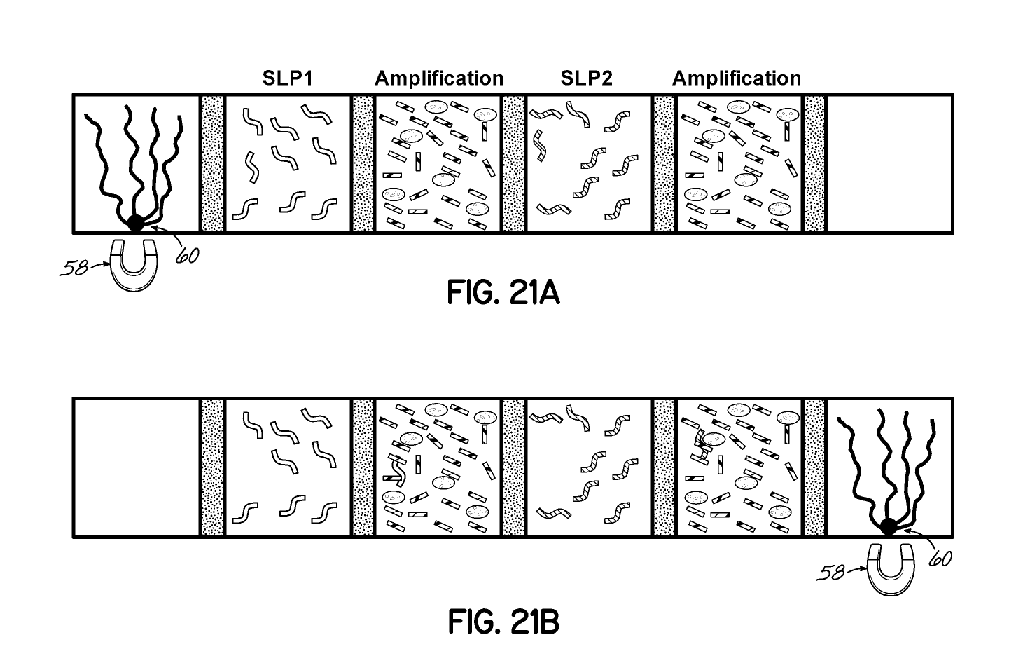

Referring to FIGS. 21A and 21B, the universal nature of the primer probe in DSPA and its use in multi-well diagnostics is shown. As described above, the bifunctional nature of RT-PCR probes requires that a fluorophore-quencher pair be attached to each DNA probe sequence, which makes quantification impractical when several targets are tested. Since DSPA separates these two functions, the same reporter molecule can be used in multiwell diagnostics. And again, as shown in FIGS. 21A and 21B, a magnetic field may be used to move any target nucleic acid attached to metal beads through cassettes that include separated segments having various reaction mixtures (e.g., a first stem loop primer probe segment, an amplification segment, a second stem loop probe segment, and a second amplification segment).

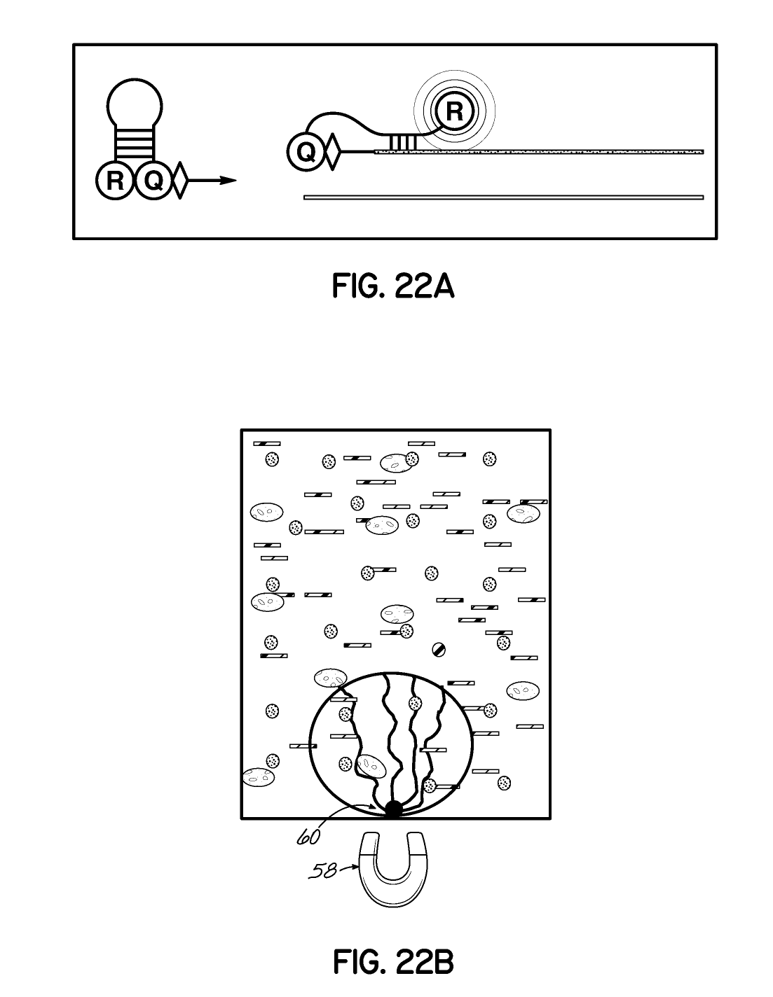

FIGS. 22A and 22B show the monomolecular nature of detection. As described above, Scorpion probes are one commonly used probe today. Scorpions use a single oligonucleotide that consists of a hybridization probe and a primer linked together via a non-amplifiable monomer. A hairpin loop contains a specific sequence that is complementary to the extension product of the primer. After replication then, the probe is covalently attached to the amplicon, which makes signal generation a monomolecular process. While this allows faster and earlier detection, Scorpions are complicated molecules having three attached modifications.

In contrast, signal generation in DSPA is not only a monomolecular reaction, but priming and probing is performed by the same oligonucleotide. Thus, DSPA allows simple detection of the very first amplicons.

The various aspects of the present invention will be described in greater detail with respect to the following nonlimiting Examples.

### Example 1

This Example describes development of primers for use in an isothermal amplification process. As described above, the primers used in various embodiments of such a process may be of a sequence that does not initially form a dissociative structure (such as a quadruplex), but that will do so upon extension of the sequence during amplification. One exemplary embodiment of such a primer sequence is G3T-ss13.

Role of Cations and Terminal Guanines in Quadruplex Formation.

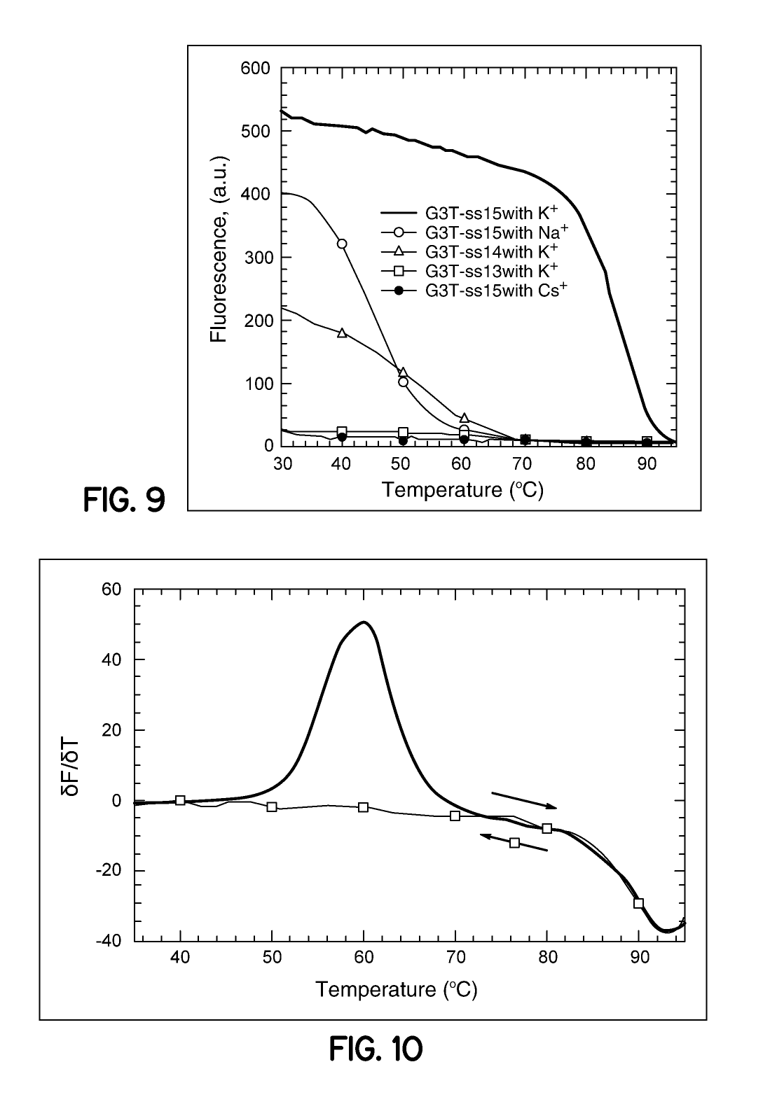

FIG. 9 demonstrates fluorescence unfolding experiments of G3T-ss15, G3T-ss14, and G35-ss13. Unfolding of G3T-ss15 was performed in the presence of 50 mM monovalent cations, Na⁺ (-∘-), K⁺ (black line) and Cs⁺ (--). In the case of Na⁺ ions the melting curve reveals the sigmoidal behavior characteristic of monophasic transition with Tₘ ˜45° C. The transition corresponds to unfolding of the quadruplex, which is accompanied by quenching of 2Ap fluorescence by adjacent guanines in the unfolded quadruplex. As expected [as shown by Jing, N., Rando, R. F., Pommier, Y. and Hogan, M. E. (1997) Ion selective folding of loop domains in a potent anti-HIV oligonucleotide, Biochemistry, 36, 12498-12505], the potassium salt of G3T-ss15 is very stable with a Tₘ of ˜88° C. (black). Thus, both Na⁺ and K⁺ ions are able to fold quadruplexes, however the latter is almost 45° C. more stable. In the presence of Cs⁺ ions G3T-ss15 does not reveal any measurable fluorescence over the entire temperature range, which suggests that Cs⁺ does not support quadruplex formation [Kankia, B. I. and Marky, L. A. (2001) Folding of the thrombin aptamer into a G-quadruplex with Sr(2+): stability, heat, and hydration, Journal of the American Chemical Society, 123, 10799-10804]. The results are in agreement with observations that K⁺ ions with ionic radii of 1.33 Å are the optimum size for a cation to enter the inner core of G-quartets, while Cs⁺ ions with ionic radii of 1.69 Å are too big [Jing, N., Rando, R. F., Pommier, Y. and Hogan, M. E. (1997) Ion selective folding of loop domains in a potent anti-HIV oligonucleotide, Biochemistry, 36, 12498-12505].

The role of terminal guanines in quadruplex formation in the presence of K⁺ ions was studied similarly. Deletion of a single guanine at the 3′-end, G3T-ss14, significantly destabilized the quadruplex (FIG. 9, -Δ-). However, it is still able to create some structure at lower temperatures. Deletion of another guanine, G3T-ss13, almost completely inhibits quadruplex formation (-□-). Thus, the experiments shown in FIG. 9 suggest that (i) in the presence of Cs⁺ ions mixing of full-length G3T-ss15 to its complementary sequence should result in a DNA duplex; and (ii) in the presence of K⁺ ions the truncated variant, G3T-ss13, should also be able to form a duplex.

Role of Cations and Terminal Guanines in Duplex Formation.

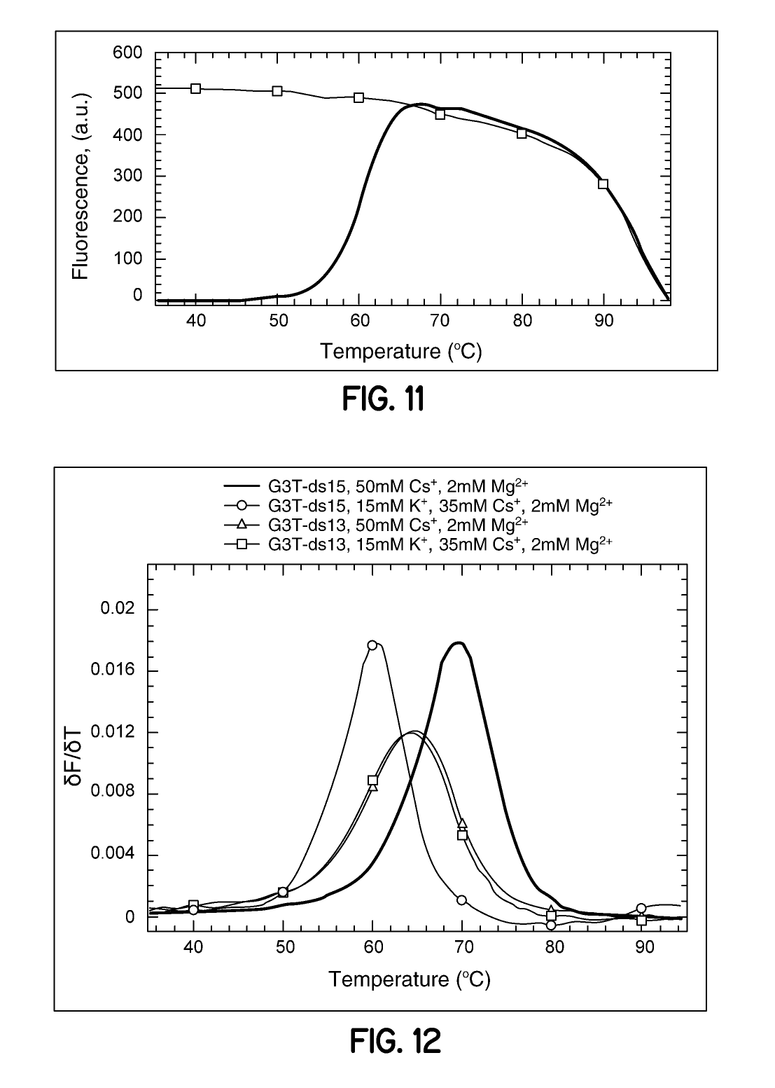

To mimic DNA conformational changes that take place upon the amplification reaction, the fluorescence melting of the G3T-ds15 duplex was studied in amplification buffer (15 mM KCl, 35 mM CsCl, 2 mM MgCl₂, 10 mM Tris-HCl, pH 8.7) (FIG. 10). To ensure that G3T-ds15 initially anneals to its complementary strand (as double-helix), the sequences were annealed in the presence of CsCl followed by later KCl addition. (K⁺ is a quadruplex forming cation, while Cs⁺ does not support quadruplexes [as described in Kankia, B. I. et al. (2001) Folding of the thrombin aptamer into a G-quadruplex with Sr(2+): stability, heat, and hydration, J Am Chem Soc, 123, 10799-10804, incorporated by reference herein in its entirety.]). The duplex is formed by annealing a shorter version of 2Ap-G3T (unable to form a quadruplex), such as G3T-ss13, to the target sequence with subsequent addition of the missing bases by Taq polymerization. The heating curve (black curve, FIGS. 10 and 11) reveals two separate transitions with midpoints at 60° C. and ˜95° C. The transition at 60° C. corresponds to duplex unfolding, which is accompanied by an increase in fluorescence due to quadruplex formation of released G3T-ss15. The second transition at ˜95° C. corresponds to the melting of the quadruplex accompanied by fluorescence quenching of 2Ap due to stacking interactions of adjacent guanines in unstructured 2Ap-G3T. The second transition is completely reversible during the cooling process (-□- in FIGS. 10 and 11). However, no duplex refolding was observed, which clearly indicates that the quadruplex stays folded at lower temperatures in the presence of the complementary strand. In separate isothermal experiments at 40° C., the complementary strand was added to a preformed G3T-ss15 quadruplex, which didn't affect the fluorescence spectrum of the quadruplex (data not shown). Thus, both melting and isothermal mixing experiments show that the quadruplex is very stable and the complementary strand is unable to invade the structure.

It is noted that the duplex melting temperature (˜60° C.) measured in the presence of the quadruplex forming cation KCl (FIGS. 10 and 11) is significantly lower than the Tₘ=70° C. of the same duplex measured under experimental conditions unfavorable for quadruplex formation (50 mM CsCl and 2 mM MgCl₂), or predicted from nearest-neighbor analysis of equilibrium unfolding [Zuker, M. (2003) Mfold web server for nucleic acid folding and hybridization prediction, Nucleic acids research, 31, 3406-3415]. To compare thermal stabilities of the G3T-ds15 duplex in the presence and absence of K+, UV absorption was employed (FIG. 12). In the presence of K+ ions, G3T-ds15 unfolds at 60° C. (-∘-), which is in excellent agreement with results of the fluorescence measurements shown in FIG. 10. In the absence of K+ ions, the duplex is significantly more stable and unfolds at 70° C. as predicted from nearest-neighbor analysis of equilibrium unfolding [Zuker, M. (2003) Mfold web server for nucleic acid folding and hybridization prediction, Nucleic acids research, 31, 3406-3415]. Note an additional small peak at 93° C. in the presence of K⁺, which corresponds to quadruplex unfolding and again agrees with fluorescence measurements shown in FIG. 10. Additional melting experiments of the G3T-ds15 duplex in the presence of K⁺ performed at slower heating rates (0.5° C./min and 0.1° C./min) further shifted the transition to lower temperatures (data not shown). Thus, in the presence of K⁺, unfolding of the duplex is a non-equilibrium process due to quadruplex formation of the released strands, which significantly destabilizes the duplex. FIG. 12 also demonstrates unfolding of G3T-ds13 in the presence and absence of K⁺ ions. Since G3T-ss13 is not able to form a quadruplex (see FIG. 9), G3T-ds13 duplex melting profiles are identical in the presence and absence of K⁺ ions with Tₘ=65° C. As expected, in the presence of Cs⁺ ions the longer duplex, G3T-ds15, is more stable than the shorter duplex, G3T-ds13. However, in the presence of K⁺ the opposite is true: the shorter duplex is ˜5° C. more stable than the longer one. This result illustrates the potential for isothermal amplification; at appropriate temperatures, the primer is more stable before elongation, which facilitates primer dissociation and the next priming round without the need for thermal denaturation.

As a result, a dissociative conformation is assumed and signal evolves after adding two guanines to a primer, such as G3T-ss13. Due to the fact that high amounts of dNTP (˜0.5 mM) may inhibit Taq, the reaction shown in FIG. 4, panel B, which only requires a two-guanine extension in the described embodiment, may allow very high concentrations of signal molecules to form, such that the signal may be detected by the unaided eye.

Further, as shown in FIG. 12, in the presence of K⁺ ions, the DNA duplex, G3T-ds13 is ˜5° C. more stable than full length sequence, G3T-ds15. Therefore, one might predict that the primer in FIG. 4 will displace the first sequence segment of the stem-loop and initiate polymerization in the absence of target nucleic acid. However, this is unlikely since the stem-loop is a monomolecular structure, which makes it entropically more favorable than the bimolecular complex formed by the primer and the stem-loop. For instance, a monomolecular 17-bp duplex is 20° C. more stable than the corresponding 15-bp bimolecular duplex. In addition, a minimum amount of K⁺ (for instance, 2 mM) may be used to further increase stem-loop stability, and increase total ionic strength in reaction buffers to avoid stem-loop unfolding by accidental temperature increase.

### Example 2

Non-Enzymatic Amplification.

In this prophetic example, the DNA stem-loop 5′GGGAGGGCGGGTGGG(T)₁₄GGCCCGCCCTC (underline=quadruplex forming sequence, bold=loop, italic=stem) [SEQ. ID. NO. 4] will be studied in the absence and the presence of target sequence, 5′CC(A)₁₄CCCA [SEQ. ID. NO. 5]. The estimated Tₘs and free energies are: 51° C. and −8 kcal/mol for the stem-loop and 72° C. and −15 kcal/mol for the bimolecular complex [Zuker, M. (2003) Mfold web server for nucleic acid folding and hybridization prediction. Nucleic acids research, 31, 3406-3415]. Thus, it is expected that the 20 bp target-loop complex should unfold the stem-loop structure. The 4-bp terminal segment of the dissociative-structure-forming sequence (underlined), TGGG, involved in complex formation is too short to inhibit quadruplex formation. Thus, upon stem unfolding, the quadruplex (or other dissociative structure) should form. A combination of UV and fluorescence melting experiments of stem-loop plus target will test these predictions. In the case of successful unfolding of a stem-loop and quadruplex formation, UV-unfolding should reveal only one peak at ˜50° C. Alternatively, two peaks may be observed: one for bimolecular complex melting at ˜50° C. and a second for the monomolecular stem-loop at ˜70° C. Any alternative two-peak observation may be the result of refolding of the stem-loop structure after melting of the complex, which means that the quadruplex was not folded.

Further Experimentation.

Stem-loops A and B may need to be altered to minimize the overlap between the quadruplex forming sequence and the target. In addition, loop and stem sequences also may need to be altered to shift Tₘs of the complex and the stem-loop. Next, the kinetics of target binding to and dissociation from the stem-loop will be investigated using the 2Ap fluorescence. Finally, complementary stem-loop will be designed for exponential increase of signal.

In such further experimentation, suitable DNA constructs will be designed by UV and fluorescence unfolding in the absence and presence of target molecules. Signal amplification will be monitored by fluorescence measurements of the most sensitive probe designed. Signal amplification will be monitored by the unaided eye using an appropriate excitation source.

### Example 3

FRET-Based Probes.

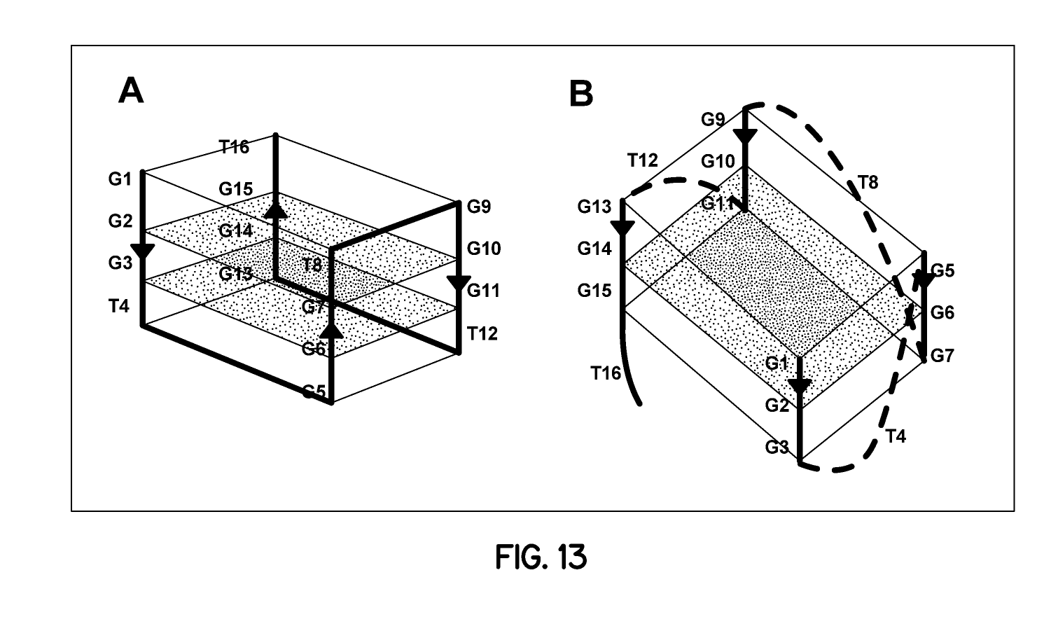

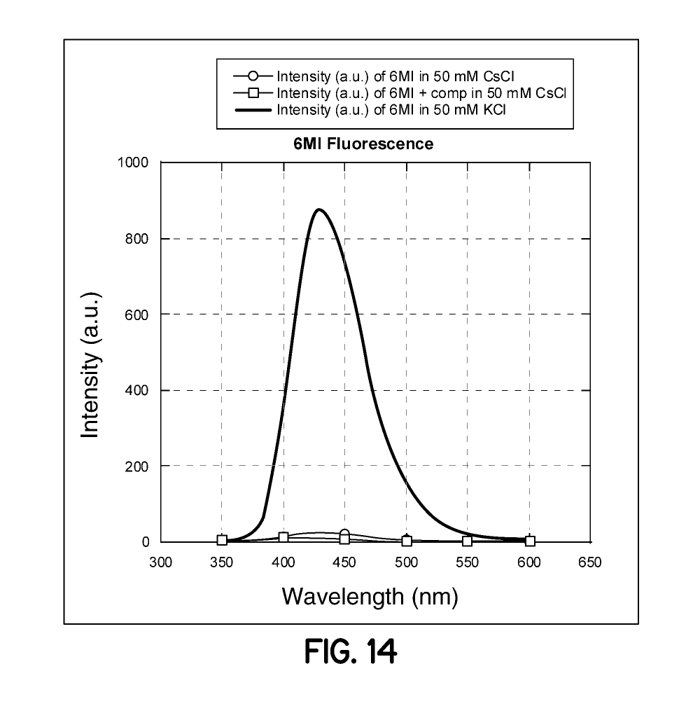

The hypothetical model of the parallel structure of a quadruplex shown in FIG. 13, panel B is based on thermodynamic and spectroscopic studies. Three G-quartets were assumed because of higher thermal stability of the G3T-ss15 quadruplex when compared with the quadruplexes with two G-quartets [Kankia, B. I. and Marky, L. A. (2001) Folding of the thrombin aptamer into a G-quadruplex with Sr(2+): stability, heat, and hydration. Journal of the American Chemical Society, 123, 10799-10804; Hardin, C. C., Perry, A. G. and White, K. (2000) Thermodynamic and kinetic characterization of the dissociation and assembly of quadruplex nucleic acids. Biopolymers, 56, 147-194]. However, two G-quartets in the G3T-ss15 sequence with three diagonal GT loops cannot be excluded. To test this possibility, substitution at positions 3, 7 and 11 will be made and studied for their effect on quadruplex formation. Depending on the outcome, incorporation of 2Ap in positions 3, 7 and 11 will also be tested.

The sensitivity of the probes will be estimated by fluorescence measurements before (quenched state) and after (emitted state) adding K⁺ ions and before (quenched) and after (emitted) adding missing guanines. Multiplexing capability will be tested by actual amplification of various segments of a plasmid DNA using four different primers with different fluorescence properties. Suitable primers for multiplexing will be designed using UV-melting experiments.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is:

1. A nucleic acid construct comprising first, second and third sequence segments, wherein:
at least a portion of the first sequence segment includes a sequence adapted to conform into a structure that dissociates from a complementary strand of a DNA duplex;
at least a portion of the second sequence segment includes a sequence that is complementary to a target nucleic acid; and
at least a portion of the third sequence segment includes a sequence that is complementary to the portion of the first sequence segment that is adapted to conform into a structure that dissociates from a complementary strand of a DNA duplex.

2. The nucleic acid construct of claim 1, wherein the first sequence segment is positioned proximal to the 5′ end of the nucleic acid.

3. The nucleic acid construct of claim 1, wherein the first sequence segment is positioned proximal to the 3′ end of the nucleic acid.

4. The nucleic acid construct of claim 1, wherein the nucleic acid is of a stem-loop structure wherein the stem comprises the first and third sequence segments in a DNA duplex.

5. The nucleic acid of claim 4, wherein the loop comprises the second sequence segment.

6. A mixture of nucleic acid constructs, comprising:
a first nucleic acid construct including a first sequence segment and a second sequence segment, wherein at least a portion of the first sequence segment includes a sequence adapted to conform into a structure that dissociates from a complementary strand of a DNA duplex, and wherein at least a portion of the second sequence segment includes a sequence that is complementary to a target nucleic acid; and
a second nucleic acid construct including a first sequence segment and a second sequence segment, wherein at least a portion of the first sequence segment includes a sequence adapted to conform into a structure that dissociates from a complementary strand of a DNA duplex, and wherein at least a portion of the second sequence segment includes a sequence that is substantially similar to the target nucleic acid such that the second sequence segment of the second nucleic acid construct can bind with the second sequence segment of the first nucleic acid construct.

7. The mixture of nucleic acid constructs of claim 6, wherein each of the first and second nucleic acid constructs has a stem-loop structure.

8. The mixture of nucleic acid constructs of claim 7, wherein at least a portion of the stem of the first nucleic acid construct comprises the first sequence segment of the first nucleic acid construct, and wherein at least a portion of the stem of the second nucleic acid construct comprises the first sequence segment of the second nucleic acid construct.

9. The mixture of nucleic acid constructs of claim 8, wherein the first sequence segment of the first nucleic acid construct is located proximal to the 5′ end of the first nucleic acid construct.

10. The mixture of nucleic acid constructs of claim 8, wherein the first sequence segment of the second nucleic acid construct is located proximal to the 5′ end of the second nucleic acid construct.

11. The mixture of nucleic acid constructs of claim 8, wherein at least a portion of the loop of the first nucleic acid construct comprises the second sequence segment of the first nucleic acid construct.

12. The mixture of nucleic acid constructs of claim 8, wherein at least a portion of the loop of the second nucleic acid construct comprises the second sequence segment of the second nucleic acid construct.

13. The mixture of nucleic acid constructs of claim 6, wherein the first sequence segment of the first nucleic acid construct includes at least two successive guanine residues proximal the 3′ end of the first sequence segment.

14. A primer for amplification of a target nucleic acid, the primer adapted to conform into a conformation having a free energy with more favorable thermodynamics than a corresponding B-DNA duplex during an extension step of a polymerase chain reaction, wherein the primer includes a fluorescent nucleotide donor and a fluorescent acceptor.

15. The primer of claim 14, wherein the fluorescent acceptor is positioned proximal to the 5′ end of the primer.

16. The primer of claim 14, wherein the fluorescent nucleotide donor is 2Ap.

17. The primer of claim 16, wherein the fluorescent acceptor is Alexa405.

18. A nucleic acid construct for detection of a target nucleic acid, the nucleic acid construct comprising:
a) a first sequence strand of nucleotides, the first sequence strand including from the 5′ to the 3′ end:
i) a first segment having a sequence of nucleotides complementary to a target nucleic acid; and
ii) a plurality of segments, each of said plurality of segments having a detectable label;
iii) wherein each of said plurality of segments is adapted to conform into a conformation having a free energy with more favorable thermodynamics than a corresponding B-DNA duplex; and
b) a plurality of nucleotide segments, wherein each of the plurality of nucleotide segments includes a sequence that is complementary to either:
i) a sequence spanning the first segment and one of the plurality of segments of the first sequence strand; or
ii) at least two of the plurality of segments of the first sequence strand.

19. The nucleic acid construct of claim 18, wherein the detectable label is a fluorescent label.

20. The nucleic acid construct of claim 19, wherein the fluorescent label is chosen from 2Ap and 6MI.

21. The nucleic acid construct of claim 18, wherein at least one of the plurality of segments includes the sequence GGGNAGGGNGGGNGGG, wherein N is a fluorescent nucleotide.

22. The nucleic acid construct of claim 21, wherein each of the plurality of segments includes the sequence GGGNAGGGNGGGNGGG.

23. The nucleic acid construct of claim 18, wherein the first sequence strand further comprises at least one repeating sequence of nucleotides chosen from T and C, wherein the at least one repeating sequence is positioned between two of the plurality of segments having a detectable label.

24. The nucleic acid construct of claim 23, wherein the first sequence strand further comprises a plurality of repeating sequences of nucleotides chosen from T and C, wherein each repeating sequence of the plurality of repeating sequences is positioned between two of the plurality of segments having a detectable label.

25. The nucleic acid construct of claim 18, wherein one end of the first sequence strand is attached to a solid surface.

26. The nucleic acid construct of claim 25, wherein the solid surface is part of a DNA chip.

27. A nucleic acid construct comprising first, second and third sequence segments, wherein:
at least a portion of the first sequence segment includes a sequence that is complementary to a primer adapted to conform into a structure that dissociates upon extension from the first sequence segment;
at least a portion of the second sequence segment includes a sequence that is complementary to a target nucleic acid; and
at least a portion of the third sequence segment includes a sequence that is complementary to a portion of the first sequence segment, and wherein the third sequence segment includes a sequence that inhibits the third sequence segment from conforming into a structure that dissociates from a complementary strand of a DNA duplex.

28. A reaction mixture comprising:
a plurality of primers, wherein each primer belongs to a single category of primer;
a polymerase;
a plurality of dNTPs; and
a plurality of nucleic acid constructs, wherein each nucleic acid construct comprises first, second and third sequence segments, wherein:
(i) at least a portion of the first sequence segment includes a sequence that is complementary to a primer adapted to conform into a structure that dissociates upon extension from the first sequence segment;
(ii) at least a portion of the second sequence segment includes a sequence that is complementary to a target nucleic acid; and
(iii) at least a portion of the third sequence segment includes a sequence that is complementary to a portion of the first sequence segment, and wherein the third sequence segment includes a sequence that inhibits the third sequence segment from conforming into a structure that dissociates from a complementary strand of a DNA duplex.
